SEQUENCE LISTING (37 sequences)

SEQ ID NO: 1 - protein, 456 aa, Lolium perenne
MKEKGGSRAAVDERYAQWKSLIPVLYDWFANHNLVWPSLSCRWGPQFEQATYKNRQRLYLSEQTDGSVPNTLVIANCEVVKPRVAAAEHISQFNEEARSPFVKKYKTIIHPGEVNRIRELPQDSRIIATHTDSPDVLIWDVEAQPNRHAVLGATDSRPDLILRGHEENAEFALAMCPAEPYVLSGGKDKSVVWWSIQDHISGLGDSSKSETSPGASGSKHSKTANEKDSPKVDPRGVFHGHDSTVEDVQFCPSSAQEFCSVGDDACLILWDARTGTSPAVKVEKAHGGDVHCVDWNLHDVNYILTGSADNSVRMWDRRNLGPGGGAGSPIHKFEGHKAAVLCVQWSPDKASVFGSSAEDGFLNVWDHDRVGKKKNPNSPGGLFFQHAGHRDKIVDFHWNSSDPWTIVSVSDDGESTGGGGTLQIWRMSDLIYRPEEEVLSELENFKSHLASCTPRA

SEQ ID NO: 2 - protein, 453 aa, Oryza sativa
MKEKGSRAAVDERYAQWKSLIPVLYDWFANHNLVWPSLSCRWGPQFEKATYKNRQRLYLSEQTDGTVPNTLVIANCEVVKPRVAAAEHISQFNEEARSPFVKKYKTIIHPGEVNRIRELPQNSKIIATHTDSPDVLIWDVEAQPNRQAQLAQMESRPDLILRGHKDIAEFALAMCPAEPYVLSGGKDKSVVWWSIQDHISALGDSSKTESSPGASGSKGKTANDKDSPKVDPRGIFLGHDSTVEDVQFCPSSAQEFCSVGDDSCLILWDARSGTGPAVKVEKAHGGDVHCVDWNLHDVNYILTGSADNSVRMWDRRNLGSGGAGIPVHKFEGHKAAVLCVQWSPDKASVFGSSAEDGFLNVWDHEKVGNKKNPNAPAGLFFQHAGHRDKIVDFHWNSSDPWTIVSVSDDGESTGGGGTLQIWRMSDLIYRPEDEVLAELENFKTHLASCAPRS

SEQ ID NO: 3 - protein, 453 aa, Zea mays
MKERGGSSAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLIWDVEAQPNRHAVLGASESRPDLILTGHKENAEFALAMCPAEPYVLSGGKDKSVVLWSIQDHISALGDSSSSPGASGSKQSIKTANEKESPKVDPRGIFHGHDSTVEDVQFCPSSAQEFCSVGDDACLILWDARTGTAPAVKVEKAHSGDVHCVDWNPLDVNYILTGSADNSVRMWDRRNLGSGGAGSPIHKFEGHKAAVLCVQWSPDRASVFGSSAEDGFLNVWDHEKVGKKKNSNVPAGLFFQHAGHRDKIVDFHWNSSDPWTIVSVSDDGESTGGGGTLQIWRMSDLIYRPEDEVLAELENFKAHLASCAPKN

SEQ ID NO: 4 - protein, 453 aa, Zea mays
MKERSGSRAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLIWDVEAQPNRHAVLGASESRPDLILTGHKENAEFALAMCPAEPYVLSGGKDKSVVLWSIQDHISALGDSSSSPGASGSKQSIKTANEKESPKVDPRGIFHGHDSTVEDVQFCPSSAQEFCSVGDDACLILWDARTGTAPAVKVEKAHSGDVHCVDWNPLDVNYILTGSADNSVRMWDRRNLGSGGAGSPIHKFEGHKAAVLCVQWSPDRASVFGSSAEDGFLNVWDHEKVGKKKNSNVPAGLFFQHAGHRDKIVDFHWNSSDPWTIVSVSDDGESTGGGGTLQIWRMSDLIYRPEDEVLAELENFKAHLASCAPKN

SEQ ID NO: 5 - protein, 453 aa, Zea mays
MKERGGSRAAVDERYAQWKSLIPVLYDWFANHNLVWPSLSCRWGPQFEKATYKNRQRLYLSEQTDGSVPNTLVIANCEVVKPRVAAAEHISQFNEEARSPFVKKYKTIVHPGEVNRIRELPQNSKIIATHTDSPDVLVWDVEAQPNRHAVLGASESRPDLILTGHQENAEFALAMCPAEPYVLSGGKDKFVVLWSIQDHISALGDSSSSPGASGSKQSGKIANEKESPKVDPRGIFHGHDSTVEDVQFCPSSAQEFCSVGDDACLILWDARTGTDPAVKVEKAHSGDVHCVDWNPLDVNYILTGSADNSVRMWDRRNLGSGGAGSPIHKFEGHKAAVLCVQWSPDRASVFGSSAEDGFLNVWDHEKVGKKKNSNVPAGLFFQHAGHRDKIVDFHWNSSDPWTIVSVSDDGESTGGGGTLQIWRMSDLIYRPEDEVLTELENFKAHLASCAPRN

SEQ ID NO: 6 - protein, 515 aa, Pisum sativum
METPPSQQGVVKKKETRGRKPKPKDDQQQTPAKTLKEAKKAQLQQQQQQQLQQQQQQQQHQASVDEKYTQWKSLVPVLYDWLANHNLVWPSLSCRWGPQLEQATYKNRQRLYLSEQTDGSVPNTLVIANCEVVKTRVAAAEHISQFNEEARSPFVKKYKTIIHPGEVNRIRELPQNSKIVATHTDSPDVLIWDVESQPNRHAVLGATNSRPDLILTGHQDNAEFALAMCPTEPYVLSGGKDKTVVLWSIEDHVTSAATDKSGGSIIKPNSKSGEGNDKTVDSPSVGPRGIYSGHDDTVEDVAFCPSSAQEFCSVGDDSCLILWDARVGSSPVVKVEKAHNADLHCVDWNPHDDNLILTGSADNSVRLFDRRNLTSNGVGSPIHKFEAHKAAVLCVQWSPDKSSVFGSSAEDGLLNIWDYEKVGKKIERAGKTINSPPGLFFQHAGHRDKVVDFHWNAYDPWTIVSVSDDCESTGGGGTLQIWRMSDLLYRPEDEVLAELEKFKSHVVACAAKTDT

SEQ ID NO: 7 - protein, 507 aa, Arabidopsis thaliana
MESDEAAAVSPQATTPSGGTGASGPKKRGRKPKTKEDSQTPSSQQQSDVKMKESGKKTQQSPSVDEKYSQWKGLVPILYDWLANHNLVWPSLSCRWGPQLEQATYKNRQRLYLSEQTDGSVPNTLVIANCEVVKPRVAAAEHISQFNEEARSPFVKKYKTIIHPGEVNRIRELPQNSKIVATHTDSPDVLIWDVETQPNRHAVLGAANSRPDLILTGHQDNAEFALAMCPTEPFVLSGGKDKSVVLWSIQDHITTIGTDSKSSGSIIKQTGEGTDKNESPTVGPRGVYHGHEDTVEDVAFSPTSAQEFCSVGDDSCLILWDARTGTNPVTKVEKAHDADLHCVDWNPHDDNLILTGSADNTVRLFDRRKLTANGVGSPIYKFEGHKAAVLCVQWSPDKSSVFGSSAEDGLLNIWDYDRVSKKSDRAAKSPAGLFFQHAGHRDKVVDFHWNASDPWTIVSVSDDCETTGGGGTLQIWRMSDLIYRPEEEVVAELEKFKSHVMTCASKP

SEQ ID NO: 8 - protein, 472 aa, Silene latifolia
MTEKGKGGKKALSVDEKYSQWKSLVPILYDWLANHNLVWPSLSCRWGPLIEEATYKNRQRLYLSEQTDGSVPNTLVIANVEVVKPRVAAAEHISKFNEEARSPFVRKFKTIIHPGEVNRIRELPQNSNIVATHTDSPDVYIWDLESQPNRPANLGTPASRPDLTLTGHQDNAEFALAMCSSEPLVLSGGKDKSVVLWSIHDHISTLATEPGSAKSPNSGSNIKKAGNGNSDNPSIGPRGIYLGHEDTVEDVQFCPSSAQQFCSVGDDSCLILWDARAGLTPVTKVEKAHNADLHCVDWNPHDENLILTGSADSSINLFDRRNLTASGVGSPVHKFQGHDAPVLCVQWSPHNRSIFGSAAEDGLLNIWDYEKVSKMETESGGKKSNHPAGLFFRHAGHRDKVVDFHWNSIDPWTLVSVSDDCSSSAGGGTLQIWRIIDLLYRPEEEVLAELDKFRSHVAACSPTPTKDVNHSA

SEQ ID NO: 9 - protein, 473 aa, Silene latifolia
EMTEKGKGGKKALSVDEKYSQWKSLVPILYDWLANHNLVWPSLSCRWGPLIEEATYKNRQRLYLSEQTDGSVPNTLVIANVEVVKPRVAAAEHISKFNEEARSPFVRKFKTIIHPGEVNRIRELPQNSNIVATHTDSPDVYIWDLESQPNRPANLGTPASRPDLTLTGHQDNAEFALAMCSSEPLVLSGGKDKSVVLWSIHDHISTLATEPGSAKSPNSGSNIKKAGNGNSDNPSIGPRGIYLGHEDTVEDVQFCPSSAQQFCSVGDDSCLILWDARAGLTPVTKVEKAHNADLHCVDWNPHDENLILTGSADSSINLFDRRNLTASGVGSPVHKFQGHDAPVLCVQWSPHNRSIFGSAAEDGLLNIWDYEKVSKMETESGGKKSNHPAGLFFRHAGHRDKVVDFHWNSIDPWTLVSVSDDCSSSAGGGTLQIWRIIDLLYRPEEEVLAELDKFRSHVAACSPTPTKDVNHSA

SEQ ID NO: 10 - protein, 40 aa, Lolium perenne
AAVDERYAQWKSLIPVLYDWFANHNLVWPSLSCRWGPQFE

SEQ ID NO: 11 - protein, 41 aa, Lolium perenne
VPNTLVIANCEVVKPRVAAAEHISQFNEEARSPFVKKYKTI

SEQ ID NO: 12 - DNA, 1371 bp, Lolium perenne
atgaaggagaagggcgggtccagggcggcggtggacgagcggtacgcgcagtggaagtcgctcatccccgtcctctacgactggttcgccaaccacaacctcgtctggccctccctctcctgccggtggggtcctcagttcgagcaggctacctacaagaaccgtcagcgcctttacctgtcagagcagaccgatggaagtgtccctaacaccctggtcattgcaaactgtgaagttgtcaagccacgggttgcagctgcagaacacatctcacagttcaatgaggaagcacgatcaccttttgtaaagaagtacaagactatcattcatcctggagaggttaaccgcatcagggagcttccacaggacagcaggatcatcgccactcatacggacagtccagatgtgcttatttgggatgttgaggctcagccaaatagacatgccgtcttaggagcaactgattctcgccctgatttgatattaagaggacacgaggaaaatgctgagtttgcccttgccatgtgtccagcggaaccatatgtgctctcaggaggaaaggacaagtcagtggtctggtggagcatccaagatcacatatctggacttggagattcttcaaaaagtgagacttccccaggggcatcaggcagcaagcatagtaaaactgcaaacgaaaaggatagtcctaaagttgatcctcgaggtgtattccacgggcatgatagcactgttgaagatgttcagttctgcccttcaagtgcacaggagttctgtagtgtgggtgatgatgcttgtcttattctttgggatgctcggactggtaccagcccagctgttaaggttgagaaagctcatggtggagatgttcattgtgtcgattggaatctccatgatgttaattatatcttaactggttctgctgataactcagtccgtatgtgggaccgccgaaatttgggtcctggaggaggagctggttctccaattcacaaattcgagggccataaagctgctgtgctttgtgttcagtggtctcctgacaaagcatctgtgtttggaagctctgcagaagatggtttcttaaatgtgtgggatcacgacagggttgggaagaagaaaaatcccaattcacctggcggacttttcttccaacatgcaggtcatagggacaagattgtagacttccactggaactcatcagatccttggaccattgtgagtgtttctgatgatggcgagagcactggtggaggtggaactttgcagatatggcgtatgagtgatctgatctaccgcccggaagaagaggttctttcagagctggagaacttcaagtctcacttggccagctgcactccaagggcttga

SEQ ID NO: 13 - DNA, 2766 bp, Lolium perenne
  misc_feature (1104)..(1109): n is a, c, g, or t
gtctgttatcattttccagttcacccccaacctaggatactctctactttactgccacccgggggacactgcgagcagagcgggaggcagacaggaacagcccagagacgagctcctcccctcccacacagagcgagcgagagagagagaggcccggagcagtcgccggcggcggccaccagcggcgcggagatgaaggagaagggcgggtccagggcggcggtggacgagcggtacgcgcagtggaagtcgctcatccccgtcctctacgactggttcgccaaccacaacctcgtctggccctccctctcctgccggtaatcctcgcctcctcccctctccccagccttctccatttccaccgttcagcggccgcagccgttttctccagtagattcatttgggggttagggttttggggccacgggaccgtttctagggttttgccttgtgcatgtgcagcctgcggtggctgtggctccagatccgtggcgtggccagtgcctgggctagtatggcgagcagggcgtgaattatggggttgagttattgtagtatcgtcctagggtttttgggtctggggtttttgtggcgtgccactgtttgagatggggattcggagcttgtgtgtcgatgtaatgcttgcttcagtgaattgtttcgatgtggtggctccttctggtgccacccatcttgtaaatctagttgcctttttttatctactactagagtgatgaaatgtgattttagtaaaatactttatgagcatcggttttgtggtgcattcctactcaccgccgggtacagctaaaagcaatcatcacaaatcagtggcattctgtttgaaatcaatgctggtttaattgctcagcgtttttggcttccaatagtggttctgcaactaaaaatttgctgatctcttggtttgattgggaacaggtggggtcctcagttcgagcaggctacctacaagaaccgtcagcgcctttacctgtcagagcaggtgagtgtttgtttccacaatcagtttttttagcatctgcaactgattcttaggctctcttttacacagtgatatttttttatttgaatctaactaagcannnnnnaccgatggaagtgtccctaacaccctggtcattgcaaactgtgaagttgtcaagccacgggttgcagctgcagaacacatctcacagttcaatgaggaagcacgatcaccttttgtaaagaagtacaagactatcattcatcctggagaggttaaccgcatcagggagcttccacaggacagcaggatcatcgccactcatacggacagtccagatgtgcttatttgggatgttgaggctcagccaaatagacatgccgtcttaggagcaactgattctcgccctgatttgatattaagaggacacgaggaaaatgctgagtttgcccttgccatgtgtccagcggaaccatatgtgctctcaggaggaaaggacaagtcagtggtctggtggagcatccaagatcacatatctggacttggagattcttcaaaaagtgagacttccccaggggcatcaggcagcaagcatagtaaaactgcaaacgaaaaggatagtcctaaagttgatcctcgaggtgtattccacgggcatgatagcactgttgaagatgttcagttctgcccttcaaggtaaataggctacactatttttcttgttttccattaaggtatcacatggttattttttattttgttaacatggttttcaatggtacataagtaatagctgtcattgtgagctgctggtttgatgtctgtctgtacattggatactatgatctttatggttctcctttttgtaaataaagtgcacaggagttctgtagtgtgggtgatgatgcttgtcttattctttgggatgctcggactggtaccagcccagctgttaaggttgagaaagctcatggtggagatgttcattgtgtcgattggaatctccatgatgttaattatatcttaactggttctgctgataactcagtccgtatgtgggaccgccgaaatttgggtcctggaggaggagctggttctccaattcacaaattcgagggccataaagctgctgtgctttgtgttcagtggtctcctgacaaagcatctgtgtttggaagctctgcagaagatggtttcttaaatgtgtgggatcacgacagggttgggaagaagaaaaatcccaattcacctggcggacttttcttccaacatgcaggtcatagggacaagattgtagacttccactggaactcatcagatccttggaccattgtgagtgtttctgatgatggcgagagcactggtggaggtggaactttgcagatatggcgtatgagtgatctgatctaccgcccggaagaagaggttctttcagagctggagaacttcaagtctcacttggccagctgcactccaagggcttgaattatgtaatttgtgcaacccaatcctgtggtagctctaggtatcttagttatctgtccatttagaacattggttctgtcgtctgatgttaaacctagacttgtacccttaaggtgcttgtgagcttatttaactttctgtgtgactcaaatctctatgctctgcaaacacagtagagctgtatttctcggtagccagactattggtgtatcgggaaacattgtactggtagcttctagttggacctattgagattgcttgtgatgaatttgcgttgacgaatttggtcagtttgt

SEQ ID NO: 14 - DNA, 1362 bp, Oryza sativa
atgaaggagaaggggtcgagggcggcggtggacgagcgctacgcgcagtggaagtcgctcatcccggtgctctacgactggttcgccaaccacaacctcgtctggccgtccctctcctgccggtggggaccccagtttgagaaggctacctacaagaatcgtcagcgtctttacctttcagagcagacggatgggactgtccctaatactctggttattgcaaattgtgaagttgtcaaaccaagggttgcagctgctgaacatatatctcagttcaatgaggaagcacgatcaccttttgtaaagaagtacaagactataattcatccaggcgaggtgaacaggattagggagcttccgcagaacagtaagatcattgccactcataccgacagcccagatgttctcatttgggatgtcgaggcccagccaaatagacaagctcaactggcacagatggagtctcgtcctgatttgatattaagaggacataaggatattgctgagtttgcgcttgctatgtgcccagctgagccatatgtgttatctggaggaaaagacaaatctgttgtatggtggagcatccaagaccacatatctgcactgggagattcctcgaaaactgaatcttctccaggggcatcaggaagcaagggaaaaactgcaaatgataaggatagtcctaaagttgatcctcgtggtatctttcttggccacgacagtactgttgaagatgtccagttctgcccttctagtgcacaggagttttgtagtgtaggcgatgattcttgtcttattctttgggatgcccggagtggtacaggcccagctgttaaggttgagaaagctcatggtggtgatgttcattgtgttgactggaacctccatgatgttaactatatcttaactggttctgcggataattctgtccgtatgtgggaccggagaaatctgggttctggaggagctggcattccagttcacaaatttgagggtcataaagctgctgttctttgtgttcagtggtcacctgacaaggcatctgtatttggaagctctgcggaagacggcttcttaaatgtgtgggatcatgagaaggttggaaataagaaaaatcccaacgcacctgctgggcttttctttcaacatgctggtcatagggataagattgtagacttccactggaattcttcggatccttggactattgtgagtgtgtctgatgatggtgagagtactggtggaggtggaacattgcagatatggcgcatgagtgacctgatataccgtccagaagatgaagttcttgcagagctggagaacttcaagactcacttggccagctgtgctccaaggagctga

SEQ ID NO: 15 - DNA, 1362 bp, Zea mays
atgaaagagagaggcggctccagcgcggcggtggacgagcgctacgcgcagtggaagtcgctcatccccgtcctctacgactggtttgccaaccacaacctcgtctggccatccctctcctgccggtgggggccacagtttgagaaagctacctacaagaatcgtcagcgcctgtacctatctgaacagacggatgggagtgtgcctaatactctggttatcgcaaattgtgaagttgtgaaacctagggttgcagctgctgaacatatctcacagttcaatgaggaagcacggtcaccttttgtaaagaagtacaaaactatagttcatcctggtgaggttaacagaatcagggaacttccacagaacagtaagatcatagccactcacactgacagtccagatgtacttatttgggatgttgaagcacaaccaaatagacatgctgtcctaggagcaagcgagtctcgccctgatctgatattaacaggacataaggaaaatgcggaatttgcgcttgccatgtgtccagcagaaccatatgtcctatcaggaggaaaggacaaatctgttgtcttgtggagcatccaagaccatatatctgcccttggggattcctcgtcttctcctggagcatctggcagcaagcagtctattaaaactgcaaatgaaaaggagagccctaaagttgatcctagaggtatatttcatggacatgacagcacggttgaagatgttcagttctgcccttccagtgcgcaggagttttgtagtgtgggtgatgatgcttgtcttattctgtgggatgctcgaactggcactgccccagctgttaaggttgagaaagctcacagtggagatgttcattgtgttgattggaatccccttgatgttaactatatcttaactgggtctgccgataactctgtccgaatgtgggatcgtcggaatctgggttcaggaggagctggttctccaattcacaaatttgaaggccataaagctgctgttctttgtgttcagtggtcacctgacagagcatctgtttttggaagttctgcagaagatggtttcttaaacgtgtgggatcatgagaaggtggggaagaagaaaaattctaatgtcccagccgggcttttctttcagcacgctggtcatagggataagattgtagacttccactggaattcgtcagatccttggacaattgtcagtgtctctgatgatggcgagagcactggtggaggtggaacactgcagatttggcgcatgagtgatttgatctaccgcccagaggatgaagttcttgcagagctggagaatttcaaggctcacttggccagctgtgcaccgaagaattga

SEQ ID NO: 16 - DNA, 1362 bp, Zea mays
atgaaggagagaagcggctccagggcggcggtggacgagcgctacgcgcagtggaagtcgctcatccccgtcctctacgactggtttgccaaccacaacctcgtctggccatccctctcctgccggtgggggccacagtttgagaaagctacctacaagaatcgtcagcgcctgtacctatctgaacagacggatgggagtgtgcctaatactctggttatcgcaaattgtgaagttgtgaaacctagggttgcagctgctgaacatatctcacagttcaatgaggaagcacggtcaccttttgtaaagaagtacaaaactatagttcatcctggtgaggttaacagaatcagggaacttccacagaacagtaagatcatagccactcacactgacagtccagatgtacttatttgggatgttgaagcacaaccaaatagacatgctgtcctaggagcaagcgagtctcgccctgatctgatattaacaggacataaggaaaatgcggaatttgcgcttgccatgtgtccagcagaaccatatgtcctatcaggaggaaaggacaaatctgttgtcttgtggagcatccaagaccatatatctgcccttggggattcctcgtcttctcctggagcatctggcagcaagcagtctattaaaactgcaaatgaaaaggagagccctaaagttgatcctagaggtatatttcatggacatgacagcacggttgaagatgttcagttctgcccttccagtgcgcaggagttttgtagtgtgggtgatgatgcttgtcttattctgtgggatgctcgaactggcactgccccagctgttaaggttgagaaagctcacagtggagatgttcattgtgttgattggaatccccttgatgttaactatatcttaactgggtctgccgataactctgtccgaatgtgggatcgtcggaatctgggttcaggaggagctggttctccaattcacaaatttgaaggccataaagctgctgttctttgtgttcagtggtcacctgacagagcatctgtttttggaagttctgcagaagatggtttcttaaacgtgtgggatcatgagaaggtggggaagaagaaaaattctaatgtcccagccgggcttttctttcagcacgctggtcatagggataagattgtagacttccactggaattcgtcagatccttggacaattgtcagtgtctctgatgatggcgagagcactggtggaggtggaacactgcagatttggcgcatgagtgatttgatctaccgcccagaggatgaagttcttgcagagctggagaatttcaaggctcatttggccagctgtgcaccgaagaattga

SEQ ID NO: 17 - DNA, 1362 bp, Zea mays
atgaaggagagaggcggctccagggcggcagtggacgagcgctacgcgcagtggaagtcgctcatccccgttctctacgactggtttgccaaccacaacctcgtttggccatccctctcctgccggtgggggccacagtttgagaaagctacctacaagaatcgtcaacgcctttacctatctgaacagaccgatgggagtgtgcctaatactctggttatcgcaaactgtgaagttgtgaaaccaagggttgcagctgctgaacatatctcgcagtttaacgaggaagcacgatcaccttttgtaaagaagtacaagactatagttcatcctggtgaagttaacagaatcagggagcttccacagaacagtaagatcatagccacacacactgacagtccagatgtacttgtttgggatgttgaagcacaaccaaatagacatgctgtcttaggagcttctgaatctcgtcctgatctgatattaacgggacaccaggaaaatgcagaatttgcgcttgccatgtgtccagcagaaccatatgtcctgtcaggaggaaaggacaaatttgttgtcttgtggagcatccaagaccacatatctgcccttggggattcctcgtcttctcccggagcatctggcagcaagcagtctggcaaaattgcaaatgaaaaggagagtcctaaagttgatcctagaggtatattccatggccatgacagtactgttgaagatgttcagttctgcccttccagtgcgcaggagttttgtagtgtgggtgatgatgcttgtcttattctgtgggatgctcgaactggcactgacccagctgttaaggttgagaaagctcacagtggagatgttcattgtgttgattggaatccccttgatgttaactatatcttaactggttctgccgataactctgtccgaatgtgggatcgtcgcaatctgggttcgggaggagctggttctccaattcacaaatttgagggccataaagctgctgttctttgtgttcagtggtcacctgacagagcatctgtttttggaagttctgcagaagatggtttcttaaatgtttgggaccatgagaaggttgggaagaagaaaaattctaatgtcccagctgggcttttctttcagcatgctggtcatagggataagatcgtagacttccactggaattcgtcagatccttggacaattgtcagtgtctcagatgatggtgagagcactggtggaggtggaacactgcagatatggcgaatgagtgatttgatctaccgcccagaggatgaagttcttacagagctggagaatttcaaggctcacctggccagttgcgctccgaggaattga

SEQ ID NO: 18 - DNA, 1548 bp, Pisum sativum
atggagactcctccgtcgcagcaaggcgtagtaaagaagaaggaaactcgaggtcgaaaacctaaaccaaaagacgatcaacaacaaacacctgccaaaacactgaaagaagcgaaaaaggcgcaactacaacagcagcaacaacagcaactgcagcaacagcagcaacaacaacaacaccaagcttcggtagacgagaaatacactcaatggaagtctctcgttcccgttctctacgattggcttgcgaatcataaccttgtttggccttctctctcttgcaggtggggtcctcagcttgaacaagctacttataagaatcggcagcggctttatctttctgagcagactgatggtagtgtcccaaatactttggtgattgcgaattgtgaggttgtgaaaactagggttgcagctgcagagcatatttcacagtttaatgaggaggctcgctctccatttgttaagaagtacaagaccatcatacatcccggggaggtgaacagaattagggaattgccgcaaaattctaagatagtggctactcacacagacagccctgatgttctcatttgggatgttgaaagtcaacctaaccgtcacgctgtccttggagccacaaactctcgtccagatttgatattgaccggacaccaagacaatgctgagtttgctcttgcgatgtgcccaactgagccttatgtcctttcaggaggaaaagataaaacagtggtgttgtggagtattgaagaccatgtaacatctgctgctacggacaagtctggtggatccattatcaaaccgaactctaaatctggggaaggcaatgacaaaactgttgatagcccttctgtcgggccaagaggtatctactctgggcacgatgatactgttgaagatgtggccttttgcccttctagtgcgcaggaattctgtagtgttggagatgattcttgtctcatattatgggatgcacgtgttggctctagtcctgttgttaaggttgaaaaagctcataatgccgatcttcactgtgttgactggaatccccatgatgataatctgattcttactgggtcggcagataattctgttcgcttgtttgatcggcgcaatctcacctctaatggggttgggtctcctattcataaatttgaggctcataaagctgccgttctttgtgttcagtggtctccagacaaatcatctgtatttggaagttcagcagaagacggtctcttaaacatttgggattatgagaaggttggtaaaaagattgagcgagctggaaaaacaataaactctcctccagggttgtttttccaacatgctggtcatagagacaaagttgttgacttccattggaatgcttatgatccatggacaattgtaagtgtgtctgatgattgtgaaagtactggtggaggaggaacattgcagatatggcgcatgagtgatttgctctatagaccagaggatgaggttttggcagagttggagaaattcaaatctcacgtggtggcttgtgctgcaaagaccgatacataa

SEQ ID NO: 19 - DNA, 1524 bp, Arabidopsis thaliana
atggagagcgacgaagcagcagcagtgtctcctcaagcaacgacaccgagcggaggaaccggagcttctgggccgaagaagagaggtcggaaacctaaaaccaaggaagattctcagacgccgtcgtctcagcaacagagcgatgttaaaatgaaagaaagtgggaagaaaacgcagcagtcgccgagtgttgacgagaagtactctcagtggaaaggtctcgtccccattctctacgactggctcgctaaccataacctcgtctggccttcactctcttgcagatggggtccgcagcttgagcaagcaacctacaagaatcgccagcgtctgtacctctcagagcaaactgatggaagtgtgcccaatactttggtcatagcaaattgtgaagttgttaagccaagggttgctgcagcagagcacatttctcagttcaatgaagaagcacgttctccatttgtgaagaagtacaagaccatcattcaccctggagaggttaacagaatcagggaactcccacagaatagtaagattgttgctactcacaccgacagtcctgatgttctcatttgggatgttgaaacccaaccaaaccgtcatgctgtgcttggagctgcaaattcccgtccagatttgatactaactgggcaccaagataatgctgaatttgctcttgccatgtgcccaacggaaccctttgtgctctccggaggcaaggacaagtcagttgttttgtggagtatccaagatcacatcacaacgattgggacagattccaaatcatctggatctatcatcaaacagactggtgaaggtactgataagaatgagagtcctactgttggcccacgaggtgtatatcatggccatgaagatacagttgaagatgtggcattcagcccgacgagtgcacaagaattctgcagtgttggtgatgattcttgccttatactatgggatgcgagaactggcacaaaccctgtcacgaaggttgaaaaagcgcatgatgctgatcttcattgtgttgattggaatcctcatgacgacaatctgatcctgacagggtcagcagacaacactgtccggttgtttgatcgtaggaagcttaccgctaatggagttggttcgcctatttacaaatttgagggacacaaagctgctgttctttgtgttcagtggtctcctgataagtcatccgtctttgggagctctgcagaagatggtctcttgaacatctgggattatgacagggtcagtaagaagtctgatcgtgcagctaaaagccccgctgggctcttcttccagcatgctggtcacagggacaaagttgttgatttccactggaatgcttcagacccttggactattgtcagtgtttctgatgactgtgagactactggtggaggtggaacattgcagatatggcggatgagtgacttgatttacagaccagaagaagaagtcgtagcagaattggagaagttcaagtcgcacgttatgacttgtgcctccaagccttaa

SEQ ID NO: 20 - DNA, 1419 bp, Silene latifolia
atgacggagaaagggaaaggaggcaagaaagcattgagtgttgatgagaaatacagtcaatggaaatcacttgttcctattttatatgattggcttgccaatcataacctcgtttggccttctctctcttgcagatggggcccgttgattgaggaggcaacttacaagaaccgccaacgtctttatctctcagaacagactgatgggagtgttcctaataccctcgtgattgccaatgttgaagtcgtgaaacccagggttgctgctgcagaacatatatcgaagttcaatgaagaggctcgttctccctttgtgaggaagttcaaaacgataatacatcccggtgaggtaaacaggattagggagctcccccagaacagtaatatagttgcaactcatactgacagtccagatgtttatatttgggatctggaaagccaacctaatcgtcctgctaatttgggaacacctgcctctcgtccagatttgactctgactggtcatcaagataatgctgaattcgctcttgcaatgtgctcttctgaaccgctggtgctttctggaggaaaggataagtcagttgttttgtggagcatccatgatcatatctcaacattggcaacagaaccagggtcagcaaaatctcccaacagtggcagcaacataaagaaagctggtaatggaaattcagataatccttctattggacctcgaggcatctatctgggacatgaggatactgttgaagatgttcaattttgcccctcaagtgcacagcagttttgcagcgtgggtgatgactcctgtctcatactatgggatgcaagagctggattgacgccagtgactaaggttgagaaagctcacaatgctgatcttcactgtgttgactggaatccacatgatgaaaacctcattctaactgggtcagctgatagttctattaatttgtttgaccgccgtaatcttactgctagtggagttggatcacctgttcataaatttcagggtcatgatgctcctgtcctttgtgttcagtggtcaccgcataatagatcgatttttggcagtgctgctgaagatggcttgctaaacatatgggactatgaaaaggttagtaagatggaaacagagagcggaggtaaaaagtcaaaccatcctgcaggcttgtttttcagacatgctggacacagggacaaagtggtggacttccactggaattcgatagatccatggactctggttagtgtatcagatgactgttcaagctctgctggaggtggaacattgcagatatggcgcataatagacttgctgtacagaccagaagaggaagttttggctgagctggacaagttcagatcccatgtagccgcttgctcgcctactcctaccaaagatgttaatcattctgcctga

SEQ ID NO: 21 - DNA, 1423 bp, Silene latifolia
agagatgacggagaaagggaaaggaggcaagaaagcattgagtgttgatgagaaatacagtcaatggaaatcacttgttcctattttatatgattggcttgccaatcataacctcgtttggccttctctctcttgcagatggggcccgttgattgaggaggcaacttacaagaaccgccaacgtctttatctctcagaacagactgatgggagtgttcctaataccctcgtgattgccaatgttgaagtcgtgaaacccagggttgctgctgcagaacatatatcgaagttcaatgaagaggctcgttctccctttgtgaggaagttcaaaacgataatacatcccggtgaggtaaacaggattagggagctcccccagaacagtaatatagttgcaactcatactgacagtccagatgtttatatttgggatctggaaagccaacctaatcgtcctgctaatttgggaacacctgcctctcgtccagatttgactctgactggtcatcaagataatgctgaattcgctcttgcaatgtgctcttctgaaccgctggtgctttctggaggaaaggataagtcagttgttttgtggagcatccatgatcatatctcaacattggcaacagaaccagggtcagcaaaatctcccaacagtggcagcaacataaagaaagctggtaatggaaattcagataatccttctattggacctcgaggcatctatctgggacatgaggatactgttgaagatgttcaattttgcccctcaagtgcacagcagttttgcagcgtgggtgatgactcctgtctcatactatgggatgcaagagctggattgacgccagtgactaaggttgagaaagctcacaatgctgatcttcactgtgttgactggaatccacatgatgaaaacctcattctaactgggtcagctgatagttctattaatttgtttgaccgccgtaatcttactgctagtggagttggatcacctgttcataaatttcagggtcatgatgctcctgtcctttgtgttcagtggtcaccgcataatagatcgatttttggcagtgctgctgaagatggcttgctaaacatatgggactatgaaaaggttagtaagatggaaacagagagcggaggtaaaaagtcaaaccatcctgcaggcttgtttttcagacatgctggacacagggacaaagtggtggacttccactggaattcgatagatccatggactctggttagtgtatcagatgactgttcaagctctgctggaggtggaacattgcagatatggcgcataatagacttgctgtacagaccagaagaggaagttttggctgagctggacaagttcagatcccatgtagccgcttgctcgcctactcctaccaaagatgttaatcattctgcctga

SEQ ID NO: 22 - DNA, 11451 bp, Artificial
  Synthetic construct ORF107 binary vector
ggaattcgatatcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccccggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcccttaattaaggcgcgccgatactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaccaggccaggtttaacatcagacgacagaaccaatgttctaaatggacagataactaagatacctagagctaccacaggattgggttgcacaaattacataattcaagcccttggagtgcagctggccaagtgagacttgaagttctccagctctgaaagaacctcttcttccgggcggtagatcagatcactcatacgccatatctgcaaagttccacctccaccagtgctctcgccatcatcagaaacactcacaatggtccaaggatctgatgagttccagtggaagtctacaatcttgtccctatgacctgcatgttggaagaaaagtccgccaggtgaattgggatttttcttcttcccaaccctgtcgtgatcccacacatttaagaaaccatcttctgcagagcttccaaacacagatgctttgtcaggagaccactgaacacaaagcacagcagctttatggccctcgaatttgtgaattggagaaccagctcctcctccaggacccaaatttcggcggtcccacatacggactgagttatcagcagaaccagttaagatataattaacatcatggagattccaatcgacacaatgaacatctccaccatgagctttctcaaccttaacagctgggctggtaccagtccgagcatcccaaagaataagacaagcatcatcacccacactacagaactcctgtgcacttgaagggcagaactgaacatcttcaacagtgctatcatgcccgtggaatacacctcgaggatcaactttaggactatccttttcgtttgcagttttactatgcttgctgcctgatgcccctggggaagtctcactttttgaagaatctccaagtccagatatgtgatcttggatgctccaccagaccactgacttgtcctttcctcctgagagcacatatggttccgctggacacatggcaagggcaaactcagcattttcctcgtgtcctcttaatatcaaatcagggcgagaatcagttgctcctaagacggcatgtctatttggctgagcctcaacatcccaaataagcacatctggactgtccgtatgagtggcgatgatcctgctgtcctgtggaagctccctgatgcggttaacctctccaggatgaatgatagtcttgtacttctttacaaaaggtgatcgtgcttcctcattgaactgtgagatgtgttctgcagctgcaacccgtggcttgacaacttcacagtttgcaatgaccagggtgttagggacacttccatcggtctgctctgacaggtaaaggcgctgacggttcttgtaggtagcctgctcgaactgaggaccccaccggcaggagagggagggccagacgaggttgtggttggcgaaccagtcgtagaggacggggatgagcgacttccactgcgcgtaccgctcgtccaccgccgccctggacccgcccttctccttcatctaatggagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagagcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggccttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctcttattaattaaggcgcgccctgca

SEQ ID NO: 23 - protein, 21 aa, Lolium perenne
LYDWLANHNLVWPSLSCRWGP

SEQ ID NO: 24 - protein, 19 aa, Lolium perenne
KTIIHPGEVNRIRELPQNS

SEQ ID NO: 25 - protein, 18 aa, Lolium perenne
ATYKNRQRLYLSEQTDGS

SEQ ID NO: 26 - protein, 24 aa, Lolium perenne
ALAMCPAEPYVLSGGKDKSVVWWS

SEQ ID NO: 27 - protein, 38 aa, Lolium perenne
PRGVFHGHDSTVEDVQFCPSSAQEFCSVGDDACLILWD

SEQ ID NO: 28 - protein, 38 aa, Lolium perenne
AVKVEKAHGGDVHCVDWNLHDVNYILTGSADNSVRMWD

SEQ ID NO: 29 - protein, 39 aa, Lolium perenne
SPIHKFEGHKAAVLCVQWSPDKASVFGSSAEDGFLNVWD

SEQ ID NO: 30 - protein, 43 aa, Lolium perenne
FHGHDSTVEDVQFCPSSAQEFCSVGDDACLILWDARTGTSPAV

SEQ ID NO: 31 - protein, 34 aa, Lolium perenne
EKAHGGDVHCVDWNLHDVNYILTGSADNSVRMWD

SEQ ID NO: 32 - protein, 34 aa, Lolium perenne
FEGHKAAVLCVQWSPDKASVFGSSAEDGFLNVWD

SEQ ID NO: 33 - protein, 138 aa, Lolium perenne
FHGHDSTVEDVQFCPSSAQEFCSVGDDACLILWDARTGTSPAVKVEKAHGGDVHCVDWNLHDVNYILTGSADNSVRMWDRRNLGPGGGAGSPIHKFEGHKAAVLCVQWSPDKASVFGSSAEDGFLNVWDHDRVGKKKN

SEQ ID NO: 34 - protein, 333 aa, Lolium perenne
EEARSPFVKKYKTIIHPGEVNRIRELPQDSRIIATHTDSPDVLIWDVEAQPNRHAVLGATDSRPDLILRGHEENAEFALAMCPAEPYVLSGGKDKSVVWWSIQDHISGLGDSSKSETSPGASGSKHSKTANEKDSPKVDPRGVFHGHDSTVEDVQFCPSSAQEFCSVGDDACLILWDARTGTSPAVKVEKAHGGDVHCVDWNLHDVNYILTGSADNSVRMWDRRNLGPGGGAGSPIHKFEGHKAAVLCVQWSPDKASVFGSSAEDGFLNVWDHDRVGKKKNPNSPGGLFFQHAGHRDKIVDFHWNSSDPWTIVSVSDDGESTGGGGTLQIWRM

SEQ ID NO: 35 - protein, 26 aa, Lolium perenne
GLGDSSKSETSPGASGSKHSKTANEK

SEQ ID NO: 36 - protein, 6 aa, Lolium perenne
GPGGGA

SEQ ID NO: 37 - protein, 10 aa, Lolium perenne
GKKKNPNSPG

(19) United States
(12) Patent Application Publication
(10) Pub. No.: US 2010/0242134 A1
(43) Pub. Date: Sep. 23, 2010

(54) METHODS AND POLYNUCLEOTIDES FOR IMPROVING PLANTS

(76) Inventors: Sathish Puthigae, Auckland (NZ); Shivendra Bajaj, Auckland (NZ); Jonathan Robert Phiillips, Chesterfield, MO (US); Catherine Jane Bryant, Auckland (NZ); Kieran Michael Elborough, Pukekohe (NZ)

Correspondence Address: GREENLEE SULLIVAN P.C., 4875 PEARL EAST CIRCLE, SUITE 200, BOULDER, CO, 80301, US

(21) Appl. No.: 12/670,643

(22) PCT Filed: Jul. 25, 2008
(86) PCT No.: PCT/NZ08/00186

§ 371 Date: May 28, 2010

Related U.S. Application Data
(60) Provisional application No. 60/952,233, filed on Jul. 26, 2007.

(51) Int. Cl. A01H 1/00; C12N 5/10; A01H 5/00; C07H 21/04; C07K 14/00; C12N 15/74; C12Q 1/68; C07K 16/00

(52) U.S. Cl. 800/278; 435/419; 800/298; 536/23.1; 530/350; 435/320.1; 435/6; 530/387.1

(57) ABSTRACT

The invention provides methods for producing a plant with altered tillering time, the methods comprising transformation of a plant with a genetic construct including a polynucleotide encoding of a polypeptide with the amino acid sequence of SEQ ID NO: 1 or a variant or fragment thereof. The method also provides isolated polypeptides, polynucleotides, constructs and vectors useful for producing a plant with altered tillering time. The method also provides plant cell and plants transformed to contain and express the polypeptides, polynucleotides and constructs. The invention also provides plants produced by methods of the invention.

### TECHNICAL FIELD

The present invention relates to compositions and methods for producing plants with altered tillering time.

### BACKGROUND ART

When a new pasture crop is sown, there is an initial lag period before the crop becomes sufficiently established and can be fully exposed to grazing animals. It would be advantageous to produce pasture crops that become established more quickly than commonly sown varieties, hence allowing grazing animals earlier access to such pasture.

Crop improvements have until recently depended on selective breeding of plants for desirable characteristics. However for many plants the heterogeneous genetic compliments produced in off-spring do not result in the same desirable traits as those of their parents, thus limiting the effectiveness of selective breeding approaches.

Advances in molecular biology now make it possible to genetically manipulate the germplasm of both plants and animals. Genetic engineering of plants involves the isolation and manipulation of genetic material and the subsequent introduction of such material into a plant. This technology has led to the development of plants that are capable of expressing pharmaceuticals and other chemicals, plants with increased pest resistance, increased stress tolerance, and plants that express other beneficial traits.

Tillering is a physiological process observed in grasses, wherein new shoots are produced from an axillary bud off a main shoot or pre-existing tiller by vegetative reproduction. Each new shoot eventually ends up as a complete unit with roots, stem, and leaves. The outcome is a dramatic increase in the number of new shoots occurring immediately adjacent to the primary shoot. The daughter tillers, like the primary shoot, will remain vegetative unless induced to flower by exposing them to appropriate vernalizing conditions. The tillering process is usually triggered by the basal buds receiving periodic exposure to sunlight. Good pasture/turf management calls for only light grazing or machine mowing several times to promote good tillering before the turf can be heavily used or the pasture can be exposed to full grazing.

Improvements in the rate of pasture establishment may be achieved by developing plants that produce tillers earlier in their development than do commonly grown varieties.

Early tillering in bulb crops, including flowers and food bulb crops as onion and garlic, may provide more time for development of bulbs of the desired size before harvest. Early tillering may also lead to earlier harvesting of crops such as chives and spring onions.

Thus, there exists a need for plants with early tillering relative to their normally cultivated counterparts.

It is an object of the invention to provide improved compositions and/or methods for developing plant varieties with altered tillering time or at least to provide the public with a useful choice.

### SUMMARY OF THE INVENTION

In a first aspect the invention provides a method for producing a plant with altered tillering time, the method comprising transformation of a plant with:
a) a polynucleotide encoding of a polypeptide with the amino acid sequence of SEQ ID NO:1 or a variant of the polypeptide, wherein the variant is capable of modulating tillering time in a plant;
b) a polynucleotide comprising a fragment, of at least 15 nucleotides in length, of the polynucleotide of a), or
c) a polynucleotide comprising a compliment, of at least 15 nucleotides in length, of the polynucleotide of a).

In one embodiment the variant has at least 70% sequence identity to a polypeptide with the amino acid sequence of SEQ ID NO: 1.

In a further embodiment the variant is derived from a plant species and comprises the amino acid sequence of SEQ ID NO: 25.

In a further embodiment the variant is derived from a monocotyledonous species and comprises the amino acid sequence of SEQ ID NO: 10.

In a further embodiment the variant is derived from a monocotyledonous species and comprises the amino acid sequence of SEQ ID NO: 11.

Preferably the variant comprises the amino acid sequence of SEQ ID NO: 10 and 11.

In a further embodiment the variant is derived from a dicotyledonous species and comprises the amino acid sequence of SEQ ID NO: 23.

In a further embodiment the variant is derived from a dicotyledonous species and comprises the amino acid sequence of SEQ ID NO: 24.

Preferably the variant comprises the amino acid sequence of SEQ ID NO: 23 and 24.

In a preferred embodiment the variant comprises an amino acid sequence selected from any one of SEQ ID NO: 2-9.

In a preferred embodiment the polynucleotide of a) encodes a polypeptide with the amino acid sequence of SEQ ID NO: 1.

Preferably the polynucleotide transformed is included in a genetic construct. Preferably the genetic construct is an expression constuct.

In a further aspect the invention provides a method of producing a plant with altered tillering time, the method comprising transformation of a plant cell or plant with:
a) a polynucleotide comprising the nucleotide sequence of SEQ ID NO: 12, or a variant thereof, wherein the variant encodes a polypeptide capable of modulating tillering time in a plant;
b) a polynucleotide comprising a fragment, of at least 15 nucleotides in length, of the polynucleotide of a), or
c) a polynucleotide comprising a complement, of at least 15 nucleotides in length, of the polynucleotide of a).

In one embodiment the variant has at least 70% sequence identity to SEQ ID NO:12.

In a further embodiment the variant comprises the sequence of any one of SEQ ID NO: 14 to 21.

In a further embodiment the variant comprises the coding sequence of any one of SEQ ID NO:14 to 21.

In a further embodiment the polynucleotide of a) comprises the sequence of SEQ ID NO: 12.

In a further embodiment the polynucleotide of a) comprises the coding sequence of SEQ ID NO: 12.

Preferably the polynucleotide transformed is included in a genetic construct. Preferably the genetic construct is an expression constuct.

Preferably the plant with altered tillering time produced by the method of the invention is early tillering relative to a suitable control plant.

In a further embodiment the method for producing a plant with altered tillering time comprises transformation of a plant cell, or plant with a genetic construct capable or altering expression of a polypeptide which modulates tillering time.

In one embodiment, the method results in a plant with delayed tillering, relative to a suitable control, due to transformation of a plant cell or plant, with a genetic construct capable of down-regulating expression of a polypeptide which promotes early tillering.

In a preferred embodiment, the method results in a plant with early tillering, relative to a suitable control, due to transformation of a plant cell or plant, with a genetic construct capable of up-regulating expression of a polypeptide which promotes early tillering.

In a further aspect the invention provides a plant cell or plant produced by a method of the invention.

Preferably the plant with altered tillering time produced by the method of the invention has early tillering relative to a suitable control plant.

In a further aspect the invention provides an isolated polynucleotide having at least 70% sequence identity to a nucleotide sequence that encodes a polypeptide comprising an amino acid sequence selected of SEQ ID NO: 1, wherein the polynucleotide encodes a polypeptide capable of modulating tillering time in a plant.

In one embodiment said nucleotide sequence comprises the sequence of SEQ ID NO: 12.

In a further embodiment said nucleotide sequence comprises the full-length coding sequence of SEQ ID NO:12.

Alternatively said nucleotide sequence comprises the sequence of SEQ ID NO: 13.

In a further aspect the invention provides an isolated polynucleotide that encodes a polypeptide comprising an amino acid sequence SEQ ID NO: 1.

In a further embodiment the polynucleotide comprises the sequence of SEQ ID NO: 12.

In a further embodiment the polynucleotide comprises the full-length coding sequence of SEQ ID NO: 12.

Alternatively the polynucleotide comprises the sequence of SEQ ID NO: 13.

In a further aspect the invention provides an isolated polynucleotide encoding a polypeptide at least 70% identity to the amino acid sequence selected of SEQ ID NO: 1, wherein the polynucleotide encodes a polypeptide capable of modulating tillering time in a plant.

In a further aspect the invention provides an isolated polynucleotide comprising the sequence of SEQ ID NO: 12 or a variant thereof, wherein the variant is from ryegrass or fescue, and encodes a polypeptide capable of modulating tillering time in a plant.

In one embodiment the isolated polynucleotide comprises the sequence of SEQ ID NO: 12.

In a further aspect the invention provides an isolated polypeptide having at least 70% sequence identity to the amino acid sequence of SEQ ID NO: 1, wherein the polypeptide is capable of modulating tillering time in a plant.

In one embodiment the isolated polypeptide of comprises the amino acid sequence of SEQ ID NO: 1.

In a further aspect the invention provides an isolated polynucleotide encoding a polypeptide of the invention.

In a further aspect the invention provides an isolated polynucleotide comprising:
a) a polynucleotide comprising a fragment, of at least 15 nucleotides in length, of a polynucleotide of the invention;
b) a polynucleotide comprising a complement, of at least 15 nucleotides in length, of the polynucleotide of the invention; or
c) a polynucleotide comprising a sequence, of at least 15 nucleotides in length, capable of hybridising to the polynucleotide of the invention.

In a further aspect the invention provides a genetic construct which comprises a polynucleotide of the invention.

In one embodiment the genetic construct is an expression construct:

In a further aspect the invention provides a genetic construct including a polynucleotide consisting of at least one of:
a) a fragment, of at least 15 nucleotides in length, of a polynucleotide of the invention;
b) a complement, of at least 15 nucleotides in length, of a polynucleotide of the invention; or
c) a sequence, of at least 15 nucleotides in length, capable of hybridising to a polynucleotide the invention.

In a further aspect the invention provides a vector comprising a genetic construct or expression construct of the invention.

In a further aspect the invention provides a host cell genetically modified to express a polynucleotide of the invention, or a polypeptide of the invention.

In a further aspect the invention provides a host cell comprising a genetic construct or expression construct of the invention.

In a further aspect the invention provides a plant cell genetically modified to express a polynucleotide of the invention, or a polypeptide of the invention.

In a further aspect the invention provides a plant cell which comprises a genetic construct of the invention or the expression construct of the invention.

In a further aspect the invention provides a plant which comprises a plant cell of the invention.

In a further aspect the invention provides a method for selecting a plant with altered tillering time relative to suitable control plant, the method comprising testing of a plant for altered expression of a polynucleotide of the invention.

In a further aspect the invention provides a method for selecting a plant with altered tillering time relative to a suitable control plant, the method comprising testing of a plant for altered expression of a polypeptide of the invention.

In a further aspect the invention provides a plant cell or plant produced by the method of the invention.

In a further aspect the invention provides a plant selected by the method of the invention.

In a further aspect the invention provides a group of plants selected by the method of the invention.

In a further aspect the invention provides an antibody raised against a polypeptide of the invention.

The polynucleotides and polynucleotide variants of the invention may be derived from any species and/or may be produced synthetically or recombinantly.

In one embodiment the polynucleotide or variant, is derived from a plant species.

In a further embodiment the polynucleotide or variant, is derived from a gymnosperm plant species.

In a further embodiment the polynucleotide or variant, is derived from an angiosperm plant species.

In a further embodiment the polynucleotide or variant, is derived from a from dicotyledonuous plant species.

In a further embodiment the polynucleotide or variant, is derived from a monocotyledonous plant species.

The polypeptide and polypeptide variants, of the invention may be derived from any species and/or may be produced synthetically or recombinantly.

In one embodiment the polypeptide or variant, is derived from a plant species.

In a further embodiment the polypeptide or variant, is derived from a gymnosperm plant species.

In a further embodiment the polypeptide or variant, is derived from an angiosperm plant species.

In a further embodiment the polypeptide or variant, is derived from a from dicotyledonous plant species.

In a further embodiment the polypeptide or variant, is derived from a monocotyledonous plant species.

The plant cells and plants, of the invention may be derived from any species.

In one embodiment the plant cell or plant, is derived from a gymnosperm plant species.

In a further embodiment the plant cell or plant, is derived from an angiosperm plant species.

In a further embodiment the plant cell or plant, is derived from a from dicotyledonous plant species.

In a further embodiment the plant cell or plant, is derived from a monocotyledonous plant species.

Preferred dicotyledonous genera include: Amygdalus, Anacardium, Anemone, Arachis, Brassica, Cajanus, Cannabis, Carthamus, Carya, Ceiba, Cicer, Claytonia, Coriandrum, Coronilla, Corydalis, Crotalaria, Cyclamen, Dentaria, Dicentra, Dolichos, Eranthis, Glycine, Gossypium, Helianthus, Lathyrus, Lens, Lespedeza, Linum, Lotus, Lupinus, Macadamia, Medicago, Melilotus, Mucuna, Olea, Onobrychis, Ornithopus, Oxalis, Papaver, Phaseolus, Phoenix, Pistacia, Pisum, Prunus, Pueraria, Ribes, Ricinus, Sesamum, Thalictrum, Theobroma, Trifolium, Trigonella, Vicia and Vigna.

Preferred dicotyledonous species include: Amygdalus communis, Anacardium occidentale, Anemone americana, Anemone occidentalis, Arachis hypogaea, Arachis hypogea, Brassica napus Rape, Brassica nigra, Brassica campestris, Cajanus cajan, Cajanus indicus, Cannabis sativa, Carthamus tinctorius, Carya illinoinensis, Ceiba pentandra, Cicer arietinum, Claytonia exigua, Claytonia megarhiza, Coriandrum sativum, Coronilla varia, Corydalis flavula, Corydalis sempervirens, Crotalaria juncea, Cyclamen coum, Dentaria laciniata, Dicentra eximia, Dicentra formosa, Dolichos lablab, Eranthis hyemalis, Gossypium arboreum, Gossypium nanking, Gossypium barbadense, Gossypium herbaceum, Gossypium hirsutum, Glycine max, Glycine ussuriensis, Glycine gracilis, Helianthus annus, Lupinus angustifolius, Lupinus luteus, Lupinus mutabilis, Lespedeza sericea, Lespedeza striata, Lotus uliginosus, Lathyrus sativus, Lens culinaris, Lespedeza stipulacea, Linum usitatissimum, Lotus corniculatus, Lupinus albus, Medicago arborea, Medicago falcate, Medicago hispida, Medicago officinalis, Medicago sativa (alfalfa), Medicago tribuloides, Macadamia integrifolia, Medicago arabica, Melilotus albus, Mucuna pruriens, Olea europaea, Onobrychis viciifolia, Ornithopus sativus, Oxalis tuberosa, Phaseolus aureus, Prunus cerasifera, Prunus cerasus, Phaseolus coccineus, Prunus domestica, Phaseolus lunatus, Prunus. maheleb, Phaseolus mungo, Prunus. persica, Prunus. pseudocerasus, Phaseolus vulgaris, Papaver somniferum, Phaseolus acutifolius, Phoenix dactylifera, Pistacia vera, Pisum sativum, Prunus amygdalus, Prunus armeniaca, Pueraria thunbergiana, Ribes nigrum, Ribes rubrum, Ribes grossularia, Ricinus communis, Sesamum indicum, Thalictrum dioicum, Thalictrum flavum, Thalictrum thalictroides, Theobroma cacao, Trifolium augustifolium, Trifolium diffusum, Trifolium hybridum, Trifolium incarnatum, Trifolium ingrescens, Trifolium pratense, Trifolium repens, Trifolium resupinatum, Trifolium subterraneum, Trifolium alexandrinum, Trigonella foenumgraecum, Vicia angustifolia, Vicia atropurpurea, Vicia cakarata, Vicia dasycarpa, Vicia ervilia, Vaccinium oxycoccos, Vicia pannonica, Vigna sesquipedalis, Vigna sinensis, Vicia villosa, Vicia faba, Vicia sative and Vigna angularis.

Preferred monocotyledonous genera include: Agropyron, Allium, Alopecurus, Andropogon, Arrhenatherum, Asparagus, Avena, Bambusa, Bellavalia, Brimeura, Brodiaea, Bulbocodium, Bothrichloa, Bouteloua, Bromus, Calamovilfa, Camassia, Cenchrus, Chionodoxa, Chloris, Colchicum, Crocus, Cymbopogon, Cynodon, Cypripedium, Dactylis, Dichanthium, Digitaria, Elaeis, Eleusine, Eragrostis, Eremurus, Erythronium, Fagopyrum, Festuca, Fritillaria, Galanthus, Helianthus, Hordeum, Hyacinthus, Hyacinthoides, Ipheion, Iris, Leucojum, Liatris, Lolium, Lycoris, Miscanthis, Miscanthus×giganteus, Muscari, Ornithogalum, Oryza, Panicum, Paspalum, Pennisetum, Phalaris, Phleum, Poa, Puschkinia, Saccharum, Secale, Setaria, Sorghastrum, Sorghum, Triticum, Vanilla, X Triticosecale Triticale and Zea.

Preferred monocotyledonous species include: Agropyron cristatum, Agropyron desertorum, Agropyron elongatum, Agropyron intermedium, Agropyron smithii, Agropyron spicatum, Agropyron trachycaulum, Agropyron trichophorum, Allium ascalonicum, Allium cepa, Allium chinense, Allium porrum, Allium schoenoprasum, Allium fistulosum, Allium sativum, Alopecurus pratensis, Andropogon gerardi, Andropogon Gerardii, Andropogon scoparious, Arrhenatherum elatius, Asparagus officinalis, Avena nuda, Avena sativa, Bambusa vulgaris, Bellevalia trifoliate, Brimeura amethystina, Brodiaea californica, Brodiaea coronaria, Brodiaea elegans, Bulbocodium versicolor, Bothrichloa barbinodis, Bothrichloa ischaemum, Bothrichloa saccharoides, Bouteloua curipendula, Bouteloua eriopoda, Bouteloua gracilis, Bromus erectus, Bromus inermis, Bromus riparius, Calamovilfa longifilia, Camassia scilloides, Cenchrus ciliaris, Chionodoxa forbesii, Chloris gayana, Colchicum autumnale, Crocus sativus, Cymbopogon nardus, Cynodon dactylon, Cypripedium acaule, Dactylis glomerata, Dichanthium annulatum, Dichanthium aristatum, Dichanthium sericeum, Digitaria decumbens, Digitaria smutsii, Elaeis guineensis, Elaeis oleifera, Eleusine coracan, Elymus angustus, Elymus junceus, Eragrostis curvula, Eragrostis tef, Eremurus robustus, Erythronium elegans, Erythronium helenae, Fagopyrum esculentum, Fagopyrum tataricum, Festuca arundinacea, Festuca ovina, Festuca pratensis, Festuca rubra, Fritillaria cirrhosa, Galanthus nivalis, Helianthus annuus sunflower, Hordeum distichum, Hordeum vulgare, Hyacinthus orientalis, Hyacinthoides hispanica, Hyacinthoides non-scripta, Ipheion sessile, Iris collettii, Iris danfordiae, Iris reticulate, Leucojum aestivum, Liatris cylindracea, Liatris elegans, Lilium longiflorum, Lolium multiflorum, Lolium perenne, Lycoris radiata, Miscanthis sinensis, Miscanthus×giganteus, Muscari armeniacum, Muscari macrocarpum, Narcissus pseudonarcissus, Ornithogalum montanum, Oryza sativa, Panicum italicium, Panicum maximum, Panicum miliaceum, Panicum purpurascens, Panicum virgatum, Panicum virgatum, Paspalum dilatatum, Paspalum notatum, Pennisetum clandestinum, Pennisetum glaucum, Pennisetum purpureum, Pennisetum spicatum, Phalaris arundinacea, Phleum bertolinii, Phleum pratense, Poa fendleriana, Poa pratensis, Poa nemoralis, Puschkinia scilloides, Saccharum officinarum, Saccharum robustum, Saccharum sinense, Saccharum spontaneum, Scilla autumnalis, Scilla peruviana, Secale cereale, Setaria italica, Setaria sphacelata, Sorghastrum nutans, Sorghum bicolor, Sorghum dochna, Sorghum halepense, Sorghum sudanense, Trillium grandiflorum, Triticum aestivum, Triticum dicoccum, Triticum durum, Triticum monococcum, Tulipa batalinii, Tulipa clusiana, Tulipa dasystemon, Tulipa gesneriana, Tulipa greigii, Tulipa kaufmanniana, Tulipa sylvestris, Tulipa turkestanica, Vanilla fragrans, X Triticosecale and Zea mays.

Preferred plants are bulb-producing plants. Preferred bulb producing plants include those from the following genera: Allium, Anemone, Arisaema, Bellevalia, Brimeura, Brodiaea, Bulbocodium, Camassia, Chionodoxa, Claytonia, Colchicum, Corydalis, Crocus, Cyclamen, Cypripedium, Dentaria, Dicentra, Eranthis, Eremurus, Erythronium, Fritillaria, Galanthus, Hyacinthus, Hyacinthoides, Ipheion, Iris, Leucojum, Liatris, Lilium, Lycoris, Muscari, Narcissus, Oxalis, Ornithogalum, Puschkinia, Scilla, Thalictrum, Trillium, and Tulipa.

Preferred bulb-producing species include: Allium ampeloprasum, Allium ampeloprasum porrum, Allium ascalonicum, Allium canadense, Allium cepa, Allium cepa aggregatum, Allium cepa ascalonicum, Allium cepa proliferum, Allium cernuum, Allium chinense, Allium fistulosum, Allium moly, Allium neapolitanum, Allium porrum, Allium sativum, Allium schoenoprasum, Allium schoenoprasum sibiricum, Allium triquetrum, Allium tuberosum, Allium ursinum, Anemone americana, Anemone occidentalis, Arisaema dracontium, Arisaema triphyllum, Bellevalia trifoliata, Brimeura amethystina, Brodiaea californica, Brodiaea coronaria, Brodiaea elegans, Bulbocodium versicolor, Camassia scilloides, Chionodoxa forbesii, Chionodoxa lucilliae, Claytonia exigua, Claytonia megarhiza, Claytonia perfoliata, Claytonia virginica, Colchicum autumnale, Colchicum luteum, Corydalis flavula, Corydalis sempervirens, Crocus sativus, Cyclamen coum, Cypripedium acaule, Cypripedium kentuckiense, Dentaria laciniata, Dicentra eximia, Dicentra formosa, Eranthis hyemalis, Eremurus robustus, Erythronium elegans, Erythronium helenae, Fritillaria cirrhosa, Fritillaria persica, Fritillaria pudica, Fritillaria recurva, Galanthus nivalis, Hyacinthus orientalis, Hyacinthoides hispanica, Hyacinthoides non-scripta, Ipheion sessile, Iris collettii, Iris danfordiae, Iris reticulate, Leucojum aestivum, Liatris cylindracea, Liatris elegans, Lilium longiflorum, Lycoris aurea, Lycoris radiata, Muscari armeniacum, Muscari macrocarpum, Narcissus pseudonarcissus, Oxalis tuberosa, Ornithogalum montanum, Puschkinia scilloides, Scilla autumnalis, Scilla peruviana, Thalictrum dioicum, Thalictrum flavum, Thalictrum thalictroides, Trillium grandiflorum, Tulipa batalinii, Tulipa clusiana, Tulipa dasystemon, Tulipa gesneriana, Tulipa greigii, Tulipa kaufmanniana, Tulipa sylvestris and Tulipa turkestanica.

Other preferred plants are forage plant species from a group comprising but not limited to the following genera: Lolium, Festuca, Dactylis, Bromus, Trifolium, Medicago, Phleum, Phalaris, Holcus, Lotus, Plantago and Cichorium.

Particularly preferred forage plants are from the genera Lolium and Trifolium. Particularly preferred species are Lolium perenne and Trifolium repens.

Other preferred plants to transform include polyploid pasture grasses. Such plants usually have low tillering ability, especially during the establishment phase. It is therefore of immense value to confer better/earlier tillering in these plants.

Preferred polyploid grasses include those from the group comprising the following genera: Lolium, Festuca, Dactylis, Bromus, Phleum, Phalaris and Holcus.

Particularly preferred polyploid grass plant species are from the genus Lolium. A particularly preferred Lolium species is Lolium perenne.

Particularly preferred monocotyledonous plant species are: Lolium perenne and Oryza sativa.

The term “plant” is intended to include a whole plant, any part of a plant, propagules and progeny of a plant.

The term ‘propagule’ means any part of a plant that may be used in reproduction or propagation, either sexual or asexual, including seeds and cuttings.

### DETAILED DESCRIPTION

### Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides that is capable of specific hybridization to a target of interest, e.g., a sequence that is at least 15 nucleotides in length. The fragments of the invention comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods of the invention.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence, that is complementary to the probe, in a hybridization-based assay. The probe may consist of a “fragment” of a polynucleotide as defined herein.

### Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides of the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides of the invention being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

### Variants

As used herein, the term “variant” refers to polynucleotide or polypeptide sequences different from the specifically identified sequences, wherein one or more nucleotides or amino acid residues is deleted, substituted, or added. Variants may be naturally occuring allelic variants, or non-naturally occurring variants. Variants may be from the same or from other species and may encompass homologues, paralogues and orthologues. In certain embodiments, variants of the inventive polypeptides and polypeptides possess biological activities that are the same or similar to those of the inventive polypeptides or polypeptides. The term “variant” with reference to polypeptides and polypeptides encompasses all forms of polypeptides and polypeptides as defined herein.

### Polynucleotide Variants

Variant polynucleotide sequences preferably exhibit at least 50%, more preferably at least 51%, more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specified polynucleotide sequence. Identity is found over a comparison window of at least 20 nucleotide positions, preferably at least 50 nucleotide positions, more preferably at least 100 nucleotide positions, and most preferably over the entire length of the specified polynucleotide sequence.

Polynucleotide sequence identity can be determined in the following manner. The subject polynucleotide sequence is compared to a candidate polynucleotide sequence using BLASTN (from the BLAST suite of programs, version 2.2.5 [November 2002]) in b12seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of b12seq are utilized except that filtering of low complexity parts should be turned off.

The identity of polynucleotide sequences may be examined using the following unix command line parameters:
b12seq -i nucleotideseq1-j nucleotideseq2 -F F -p blastn

The parameter -F F turns off filtering of low complexity sections. The parameter -p selects the appropriate algorithm for the pair of sequences. The b12seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available b12seq program from the BLAST suite of programs (version 2.2.5 [November 2002]) from NCBI (ftp://ftp.ncbi.nih.gov/blast/).

The similarity of polynucleotide sequences may be examined using the following unix command line parameters:
b12seq -i nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the b12seq program. For small E values, much less than one, the E value is approximately the probability of such a random match.

Variant polynucleotide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰ more preferably less than 1×10⁻⁸⁰, more preferably less than 1×10⁻⁹⁰, more preferably less than 1×10⁻¹⁰⁰ more preferably less than 1×10⁻¹¹⁰, and most preferably less than 1×10⁻¹²⁰ when compared with any one of the specifically identified sequences.

Alternatively, variant polynucleotides of the present invention hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C-log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at65° C.

With respect to polynucleotide molecules having a length less than 100 bases, exemplary stringent hybridization conditions are 5 to 10° C. below Tm. On average, the Tm of a polynucleotide molecule of length less than 100 by is reduced by approximately (500/oligonucleotide length)° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available b12seq program from the BLAST suite of programs (version 2.2.5 [November 2002]) from NCBI (ftp://ftp.ncbi.nih.gov/blast/) via the tblastx algorithm as previously described.

### Polypeptide Variants

The term “variant” with reference to polypeptides encompasses naturally occurring, recombinantly and synthetically produced polypeptides. Variant polypeptide sequences preferably exhibit at least 50%, more preferably at least 51%; more preferably at least 52%, more preferably at least 53%, more preferably at least 54%, more preferably at least 55%, more preferably at least 56%, more preferably at least 57%, more preferably at least 58%, more preferably at least 59%, more preferably at least 60%, more preferably at least 61%, more preferably at least 62%, more preferably at least 63%, more preferably at least 64%, more preferably at least 65%, more preferably at least 66%, more preferably at least 67%, more preferably at least 68%, more preferably at least 69%, more preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.5 [November 2002]) in b12seq, which is publicly available from NCBI (ftp://ftp.ncbi.nih.gov/blast/). The default parameters of b12seq are utilized except that filtering of low complexity regions should be turned off

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Use of BLASTP as described above is preferred for use in the determination of polypeptide variants according to the present invention.

Polypeptide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available b12seq program from the BLAST suite of programs (version 2.2.5 [November 2002]) from NCBI (ftp://ftp.ncbi.nih.gov/blast/). The similarity of polypeptide sequences may be examined using the following unix command line parameters:
b12seq -i peptideseq1 -j peptideseq2 -F F -p blastp

Variant polypeptide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰, more preferably less than 1×10⁻⁷⁰, more preferably less than 1×10⁻⁸⁰, more preferably less than 1×10⁻⁹⁰, more preferably less than 1×10⁻¹⁰⁰, more preferably less than 1×10⁻¹¹⁰, more preferably less than 1×10⁻¹²⁰ and most preferably less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

### Constructs, Vectors and Components Thereof

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

The term “noncoding region” refers to untranslated sequences that are upstream of the translational start site and downstream of the translational stop site. These sequences are also referred to respectively as the 5′ UTR and the 3′ UTR. These regions include elements required for transcription initiation and termination and for regulation of translation efficiency.

Terminators are sequences, which terminate transcription, and are found in the 3′ untranslated ends of genes downstream of the translated sequence. Terminators are important determinants of mRNA stability and in some cases have been found to have spatial regulatory functions. The terminator may be “homologous” with respect to the polynucleotide to be expressed, that is the promoter may be found operably linked to the same polynucleotide in nature. Alternatively the terminator may be “heterologous”, with respect to the polynucleotide to be expressed, that is the terminator may not be found operably linked to the same polynucleotide in nature. A heterologous terminator may be normally associated with a different gene/sequence in nature.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors. The promoter may be “homologous” with respect to the polynucleotide to be expressed, that is the promoter may be found operably linked to the same polynucleotide in nature. Alternatively the promoter may be “heterologous”, with respect to the polynucleotide to be expressed, that is the promoter may not be found operably linked to the same polynucleotide in nature. A heterologous promoter may be normally associated with a different gene/sequence in nature.

A “transgene” is a polynucleotide that is taken from one organism and introduced into a different organism by transformation. The transgene may be derived from the same species or from a different species as the species of the organism into which the transgene is introduced.

An “inverted repeat” is a sequence that is repeated, where the second half of the repeat is in the complementary strand, e.g.,

(5′)GATCTA . . . TAGATC(3′)
(3′)CTAGAT . . . ATCTAG(5′)

Read-through transcription will produce a transcript that undergoes complementary base-pairing to form a hairpin structure provided that there is a 3-5 by spacer between the repeated regions.

A “transgenic plant” refers to a plant which contains new genetic material as a result of genetic manipulation or transformation. The new genetic material may be derived from a plant of the same species as the resulting transgenic plant or from a different species.

The terms “to alter expression of” and “altered expression” of a polynucleotide or polypeptide of the invention, are intended to encompass the situation where genomic DNA corresponding to a polynucleotide of the invention is modified thus leading to altered expression of a polynucleotide or polypeptide of the invention. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

The term “tillering time” refers to the period in development of a plant during which tillers are produced. Thus a plant with early tillering produces tillers at an earlier period of development than would a suitable control. Conversely, a plant with delayed tillering produces tiller at a later period of development than would a suitable control plant.

Suitable control plants may include non-transformed plants of the same species and variety, or plants of the same species or variety transformed with a control construct.

The term “altered” with reference to tillering is intended to encompass either early or delayed tillering.

The term “modulating” with reference to tillering time is intended to encompass either producing early tillering or delaying tillering.

The invention provides methods for producing and/or selecting plants with altered tillering time relative to suitable control plants, including plants with both early and delayed tillering and plants produced by such methods.

The invention provides a polynucleotide (SEQ ID NO: 12) encoding a polypeptide (SEQ ID NO:1) which modulates tillering time in plants. The invention provides polynucleotide variants of SEQ ID NO:12 (SEQ ID NO: 13 to 21) which encode polypeptide variants of SEQ ID NO: 1 (SEQ ID NO:2 to 10). The applicants have also identified a consensus polypeptide sequence (SEQ ID NO: 11) present in SEQ ID NO: 1 to 10.

### Methods for Isolating or Producing Polynucleotides

The polynucleotide molecules of the invention can be isolated by using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser, incorporated herein by reference. The polypeptides of the invention can be amplified using primers, as defined herein, derived from the polynucleotide sequences of the invention.

Further methods for isolating polynucleotides, of the invention or useful in the methods of the invention, include use of all, or portions of, the polynucleotides set forth herein as hybridization probes. The technique of hybridizing labeled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1× Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments of the invention may be produced by techniques well-known in the art such as restriction endonuclease digestion and oligonucleotide synthesis.

A partial polynucleotide sequence may be used, in methods well-known in the art to identify the corresponding full length polynucleotide sequence. Such methods include PCR-based methods, 5′RACE (Frohman M A, 1993, Methods Enzymol. 218: 340-56) and hybridization- based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species. Variants (including orthologues) may be identified by the methods described.

### Methods for Identifying Variants

### Physical Methods

Variant polynucleotides may be identified using PCR-based methods (Mullis et al., Eds. 1994 The Polymerase Chain Reaction, Birkhauser). Typically, the polynucleotide sequence of a primer, useful to amplify variant polynucleotide molecules PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides of the invention (Sambrook et al., Molecular Cloning: A Laboratory Manual, 2nd Ed. Cold Spring Harbor Press, 1987) or by identifying polypeptides from natural sources with the aid of such antibodies.

### Computer Based Methods

Polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

### Methods for Isolating Polypeptides

The polypeptides of the invention, including variant polypeptides, may be prepared using peptide synthesis methods well known in the art such as direct peptide synthesis using solid phase techniques (e.g. Stewart et al., 1969, in Solid-Phase Peptide Synthesis, WH Freeman Co, San Francisco Calif., or automated synthesis, for example using an Applied Biosystems 431A Peptide Synthesizer (Foster City, Calif.). Mutated forms of the polypeptides may also be produced during such syntheses.

The polypeptides and variant polypeptides of the invention may also be purified from natural sources using a variety of techniques that are well known in the art (e.g. Deutscher, 1990, Ed, Methods in Enzymology, Vol. 182, Guide to Protein Purification,).

Alternatively the polypeptides and variant polypeptides of the invention may be expressed recombinantly in suitable host cells and separated from the cells as discussed below:

### Methods for Producing Constructs and Vectors

The genetic constructs of the present invention comprise one or more polynucleotide sequences of the invention and/or polynucleotides encoding polypeptides of the invention, and may be useful for transforming, for example, bacterial, fungal, insect, mammalian or plant organisms. The genetic constructs of the invention are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987 ; Ausubel et al., Current Protocols in Molecular Biology, Greene Publishing, 1987).

### Methods for Producing Host Cells Comprising Constructs and Vectors

The invention provides a host cell which comprises a genetic construct or vector of the invention. Host cells may be derived from, for example, bacterial, fungal, insect, mammalian or plant organisms.

Host cells comprising genetic constructs, such as expression constructs, of the invention are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides of the invention. Such methods may involve the culture of host cells in an appropriate medium in conditions suitable for or conducive to expression of a polypeptide of the invention. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

### Methods for Producing Plant Cells and Plants Comprising Constructs and Vectors

The invention further provides plant cells which comprise a genetic construct of the invention, and plant cells modified to alter expression of a polynucleotide or polypeptide of the invention. Plants comprising such cells also form an aspect of the invention.

Production of plants altered in tillering time may be achieved through methods of the invention. Such methods may involve the transformation of plant cells and plants, with a construct designed to alter expression of a polynucleotide or polypeptide capable of modulating tillering time in such plant cells and plants. Such methods also include the transformation of plant cells and plants with a combination of constructs designed to alter expression of one or more polypeptides or polypeptides capable of modulating tillering time in such plant cells and plants.

Methods for transforming plant cells, plants and portions thereof with polynucleotides are described in Draper et al., 1988, Plant Genetic Transformation and Gene Expression. A Laboratory Manual, Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

### Methods for Genetic Manipulation of Plants

A number of strategies for genetically manipulating plants are available (e.g. Birch, 1997, Ann Rev Plant Phys Plant Mol Biol, 48, 297). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

Genetic constructs for expression of genes in transgenic plants typically include promoters for driving the expression of one or more cloned polynucleotide, terminators and selectable marker sequences to detest presence of the genetic construct in the transformed plant.

The promoters suitable for use in the constructs of this invention are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zin gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenbert. Eds) Springer Verlag. Berline, pp. 325-336.

Gene silencing strategies may be focused on the gene itself or regulatory elements which effect expression of the encoded polypeptide. “Regulatory elements” is used here in the widest possible sense and includes other genes which interact with the gene of interest.

Genetic constructs designed to decrease or silence the expression of a polynucleotide/polypeptide of the invention may include an antisense copy of a polynucleotide of the invention. In such constructs the polynucleotide is placed in an antisense orientation with respect to the promoter and terminator.

An “antisense” polynucleotide is obtained by inverting a polynucleotide or a segment of the polynucleotide so that the transcript produced will be complementary to the mRNA transcript of the gene, e.g.,

| | |
|---|---|
| 5′GATCTA 3′ | 3′CTAGAT 5′ |
| (coding strand) | (antisense strand) |
| 3′CUAGAU 5′ | 5′GAUCUCG 3′ |
| mRNA | antisense RNA |

Genetic constructs designed for gene silencing may also include an inverted repeat. An ‘inverted repeat’ is a sequence that is repeated where the second half of the repeat is in the complementary strand, e.g.,

5′-GATCTA . . . TAGATC-3′
3′-CTAGAT . . . ATCTAG-5′

The transcript formed may undergo complementary base pairing to form a hairpin structure. Usually a spacer of at least 3-5 by between the repeated region is required to allow hairpin formation.

Another silencing approach involves the use of a small antisense RNA targeted to the transcript equivalent to an miRNA (Llave et al., 2002, Science 297, 2053). Use of such small antisense RNA corresponding to polynucleotide of the invention is expressly contemplated.

The term genetic construct as used herein also includes small antisense RNAs and other such polypeptides effecting gene silencing.

Transformation with an expression construct, as herein defined, may also result in gene silencing through a process known as sense suppression (e.g. Napoli et al., 1990, Plant Cell 2, 279; de Carvalho Niebel et al., 1995, Plant Cell, 7, 347). In some cases sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the expression of a polynucleotide of the invention is also contemplated.

The polynucleotide inserts in genetic constructs designed for gene silencing may correspond to coding sequence and/or non-coding sequence, such as promoter and/or intron and/or 5′ or 3′ UTR sequence, or the corresponding gene.

Other gene silencing strategies include dominant negative approaches and the use of ribozyme constructs (McIntyre, 1996, Transgenic Res, 5, 257)

Pre-transcriptional silencing may be brought about through mutation of the gene itself or its regulatory elements. Such mutations may include point mutations, frameshifts, insertions, deletions and substitutions.

The following are representative publications disclosing genetic transformation protocols that can be used to genetically transform the following plant species: Rice (Alam et al., 1999, Plant Cell Rep. 18, 572); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, : 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5, 846, 797 and 5,004,863); perennial ryegrass (Bajaj et al., 2006, Plant Cell Rep. 25, 651); grasses (U.S. Pat. Nos. 5,187,073, 6,020,539); peppermint (Niu et al., 1998, Plant Cell Rep. 17, 165); citrus plants (Pena et al., 1995, Plant Sci.104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. No. 5,591,616 and 6,037,522); brassica (U.S. Pat. No. 5,188,958; 5,463,174 and 5,750,871); and cereals (U.S. Pat. No. 6,074,877). Other species are contemplated and suitable methods and protocols are available in the scientific literature for use by those skilled in the art.

Several further methods known in the art may be employed to alter expression of a nucleotide and/or polypeptide of the invention. Such methods include but are not limited to Tilling (Till et al., 2003, Methods Mol Biol, 2%, 205), so called “Deletagene” technology (Li et al., 2001, Plant Journal 27(3), 235) and the use of artificial transcription factors such as synthetic zinc finger transcription factors. (e.g. Jouvenot et al., 2003, Gene Therapy 10, 513). Additionally antibodies or fragments thereof, targeted to a particular polypeptide may also be expressed in plants to modulate the activity of that polypeptide (Jobling et al., 2003, Nat. Biotechnol., 21(1), 35). Transposon tagging approaches may also be applied. Additionally peptides interacting with a polypeptide of the invention may be identified through technologies such as phase-display (Dyax Corporation). Such interacting peptides may be expressed in or applied to a plant to affect activity of a polypeptide of the invention. Use of each of the above approaches in alteration of expression of a nucleotide and/or polypeptide of the invention is specifically contemplated.

### Methods for Selecting Plants

Methods are also provided for selecting plants with altered tillering time. Such methods involve testing of plants for altered for the expression of a polynucleotide or polypeptide of the invention. Such methods may be applied at a young age or early developmental stage when altered tillering may not necessarily be visible, to accelerate breeding programs directed toward improving tillering time.

The expression of a polynucleotide, such as a messenger RNA, is often used as an indicator of expression of a corresponding polypeptide. Exemplary methods for measuring the expression of a polynucleotide include but are not limited to Northern analysis, RT-PCR and dot-blot analysis (Sambrook et al., Molecular Cloning: A Laboratory Manual, 2nd Ed. Cold Spring Harbor Press, 1987). Polynucleotides or portions of the polynucleotides of the invention are thus useful as probes or primers, as herein defined, in methods for the identification of plants with altered tillering time. The polypeptides of the invention may be used as probes in hybridization experiments, or as primers in PCR based experiments, designed to identify such plants.

Alternatively antibodies may be raised against polypeptides of the invention. Methods for raising and using antibodies are standard in the art (see for example: Antibodies, A Laboratory Manual, Harlow A Lane, Eds, Cold Spring Harbour Laboratory, 1998). Such antibodies may be used in methods to detect altered expression of polypeptides which modulate tillering time in plants. Such methods may include ELISA (Kemeny, 1991, A Practical Guide to ELISA, NY Pergamon Press) and Western analysis (Towbin & Gordon, 1994, J Immunol Methods, 72, 313).

These approaches for analysis of polynucleotide or polypeptide expression and the selection of plants with altered expression are useful in conventional breeding programs designed to produce varieties with altered tillering time.

### Plants

The plants of the invention may be grown and either selfed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also form an aspect of the present invention.

Tillering time in a plant may also be altered through methods of the invention. Such methods may involve the transformation of plant cells and plants, with a construct of the invention designed to alter expression of a polynucleotide or polypeptide which modulates tillering time in such plant cells and plants. Such methods also include the transformation of plant cells and plants with a combination of the construct of the invention and one or more other constructs designed to alter expression of one or more polynucleotides or polypeptides which modulates tillering time in plants.

Exemplary methods for assessing tillering time in plants are provided in Example 1 below.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### BRIEF DESCRIPTION OF THE DRAWINGS

The present invention will be better understood with reference to the accompanying drawings in which:

FIG. 1 shows the output summary of a BLASTP search of the UNIPROT database (release date 8.2 consists of: Swiss-Prot Protein Knowledgebase Release 50.2 of 27 Jun. 2006 and TrEMBL Protein Database Release 33.2 of 27 Jun. 2006) in which the ORF107 polypeptide sequence (SEQ ID NO:1) was used as a seed sequence.

FIG. 2 shows a “Prettyplot” alignment of polypeptides (SEQ ID NO:1 to 9) including ORF107 and variants thereof and illustrates two consensus sequence motifs (VPNTLVIANCEVVKPRVAAAEHISQFNEEARSPFVKKYKTI; SEQ ID NO: 10 and AAVDERYAQWKSLIPVLYDWFANHNLVWPSLSCRWGPQFE; SEQ ID NO: 11) present in all of the variant sequences identified in monocotyledonous species, two consensus sequence motifs (LYDWLANHNLVWPSLSCRWGP; SEQ ID NO: 23 and KTIIHPGEVNRIRELPQNS; SEQ ID NO: 24) present in all of the variant sequences identified in dicotyledonous species, and a consensus sequence motif (ATYKNRQRLYLSEQTDGS; SEQ ID NO: 25) present in all of the variant sequences identified in both monocotyledonous and dicotyledonous species.

FIG. 3 shows a “Prettyplot” alignment of polyeptides (SEQ ID NO:1-5) including ORF107 and monocot variants thereof and illustrates the position of two consensus sequence motifs (VPNTLVIANCEVVKPRVAAAEHISQFNEEARSPFVKKYKTI; SEQ ID NO: 10 and AAVDERYAQWKSLIPVLYDWFANHNLVWPSLSCRWGPQFE; SEQ ID NO: 11) present in all of the sequences.

FIG. 4 shows a map of an over-expression vector (ORF107_pacI_B DNA), for plant transformation, comprising ORF 107 cloned in sense orientation (SEQ ID NO:22).

FIG. 5 shows a DNA gel-blot analysis on genomic DNA from ORF 107 T0 transgenic plants transformed with binary ORF107_pacI_B DNA. The genomic DNA was digested with (a) HindIII and (b) EcoRI and probed with a fragment of hpt coding sequence to determine gene-integration copy number and to identify independent transformation events.

FIGS. 6a-6d show the growth parameters observed for transgenic ORF107 T1 plant lines compared to a and wild type control (Nipponbare). FIG. 6a shows plant height measurements until maturity phase. FIG. 6b shows plant tiller measurements until maturity phase. FIG. 6c shows statistical analysis of plant height measurements recorded until mid-tillering phase. FIG. 6d shows statistical analysis of plant plant tiller measurements recorded until mid-tillering phase.

### EXAMPLES

The invention will now be illustrated with reference to the following non-limiting examples.

### Example 1

### Altered Tillering Time by In-Plant Expression of a Polynucleotide of the Invention

A polynucleotide designated ORF107 (SEQ ID NO:12) was identified in a ViaLactia Biosciences Ltd proprietary ryegrass (Lolium perenne) Gene Thresher (Orion Genomics) genomic library.

ORF107 appears to encode a WD-40 repeat polypeptide (SEQ ID NO:1). WD-40 repeats (also known as WD or beta-transducin repeats) are short ˜40 amino acid motifs, often terminating in a Trp-Asp (W-D) dipeptide. WD-containing proteins have 4 to 16 repeating units, all of which are thought to form a circularised beta-propeller structure. WD-repeat proteins are a large family found in all eukaryotes and are implicated in a variety of functions ranging from signal transduction and transcription regulation to cell cycle control and apoptosis. The underlying common function of all WD-repeat proteins is coordinating multi-protein complex assemblies, where the repeating units serve as a rigid scaffold for protein interactions. The specificity of the proteins is determined by the sequences outside the repeats themselves. In the case of ORF107, the applicants have identified the motifs: GLGDSSKSETSPGASGSKHSKTANEK (SEQ ID NO:35), GPGGGA (SEQ ID NO:36) and GKKKNPNSPG (SEQ ID NO:37) in general and the residues: T211, P322 and G380 specifically, as being important for the early establishment and improved tillering phenotype observed in transgenic rice.

Motifs observed in ORF107 protein:
PF00400 WD40

(SEQ ID NO: 26)

1. ALAMCPAEPYVLSGGKDKSVVWWS

(SEQ ID NO: 27)

2. PRGVFHGHDSTVEDVQFCPSSAQEFCSVGDDACLILWD

(SEQ ID NO: 28)

3. AVKVEKAHGGDVHCVDWNLHDVNYILTGSADNSVRMWD

(SEQ ID NO: 29)

4. SPIHKFEGHKAAVLCVQWSPDKASVFGSSAEDGFLNVWD
PS50082 WD_REPEATS_2 Trp-Asp (WD) repeats profile

(SEQ ID NO: 30)

1. FHGHDSTVEDVQFCPSSAQEFCSVGDDACLILWDARTGTSPAV

(SEQ ID NO: 31)

2. EKAHGGDVHCVDWNLHDVNYILTGSADNSVRMWD

(SEQ ID NO: 32)

3. FEGHKAAVLCVQWSPDKASVFGSSAEDGFLNVWD
PS50294 WD_REPEATS_REGION Trp-Asp (WD) repeats
circular profile

(SEQ ID NO: 33)

FHGHDSTVEDVQFCPSSAQEFCSVGDDACLILWDARTGTSPAVKVEKAHG
GDVHCVDWNLHDVNYILTGSADNSVRMWDRRNLGPGGGAGSPIHKFEGHK
AAVLCVQWSPDKASVFGSSAEDGFLNVWDHDRVGKKKN
SSF50978 WD40-repeat like

(SEQ ID NO: 34)

EEARSPFVKKYKTIIHPGEVNRIRELPQDSRIIATHTDSPDVLIWDVEAQ
PNRHAVLGATDSRPDLILRGHEENAEFALAMCPAEPYVLSGGKDKSVVWW
SIQDHISGLGDSSKSETSPGASGSKHSKTANEKDSPKVDPRGVEHGHDST
VEDVQFCPSSAQEFCSVGDDACLILWDARTGTSPAVKVEKAHGGDVHCVD
WNLHDVNYILTGSADNSVRMWDRRNLGPGGGAGSPIHKFEGHKAAVLCVQ
WSPDKASVEGSSAEDGFLNVWDHDRVGKKKNPNSPGGLFFQHAGHRDKIV
DFHWNSSDPWTIVSVSDDGESTGGGGILQIWRM

ORF107 Variants

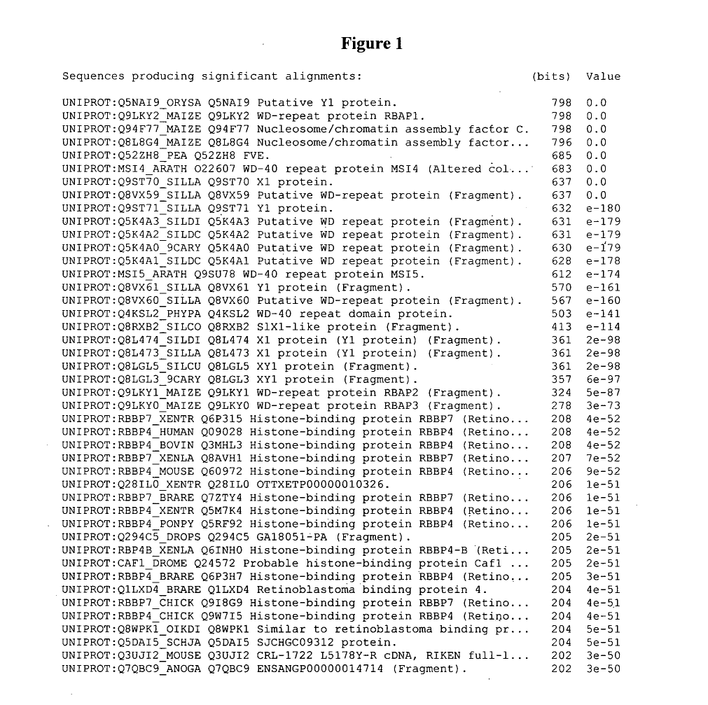

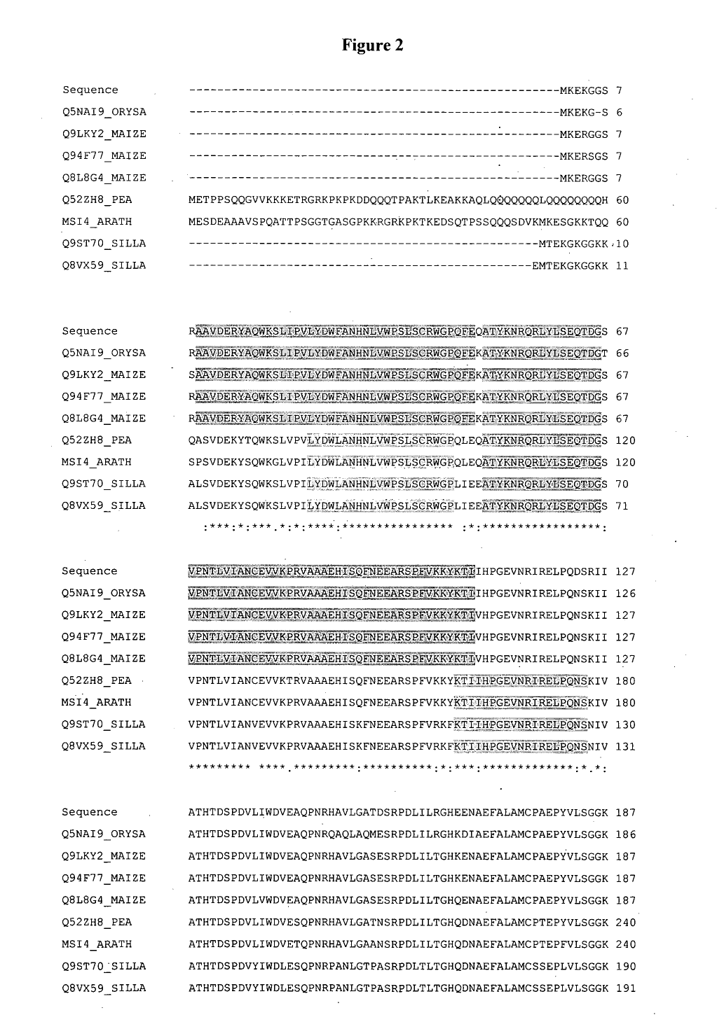

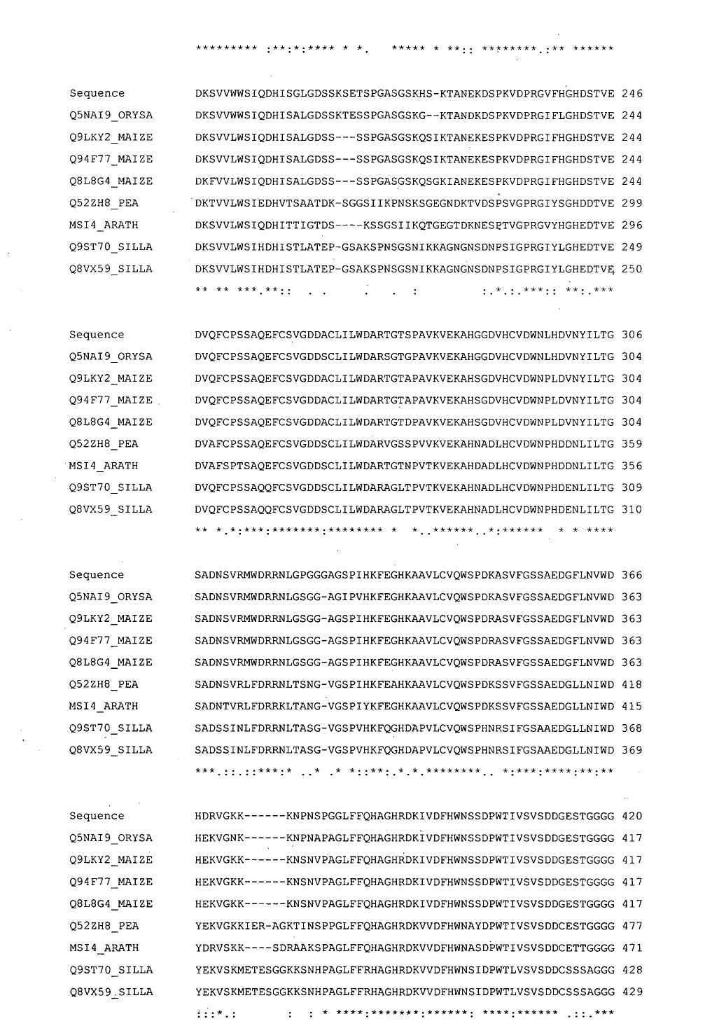

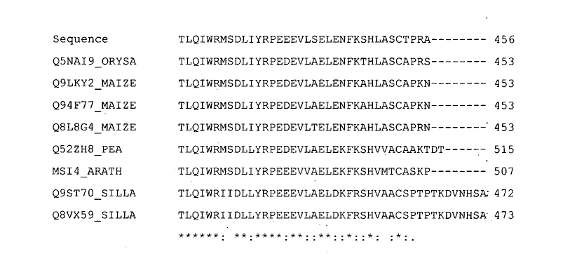

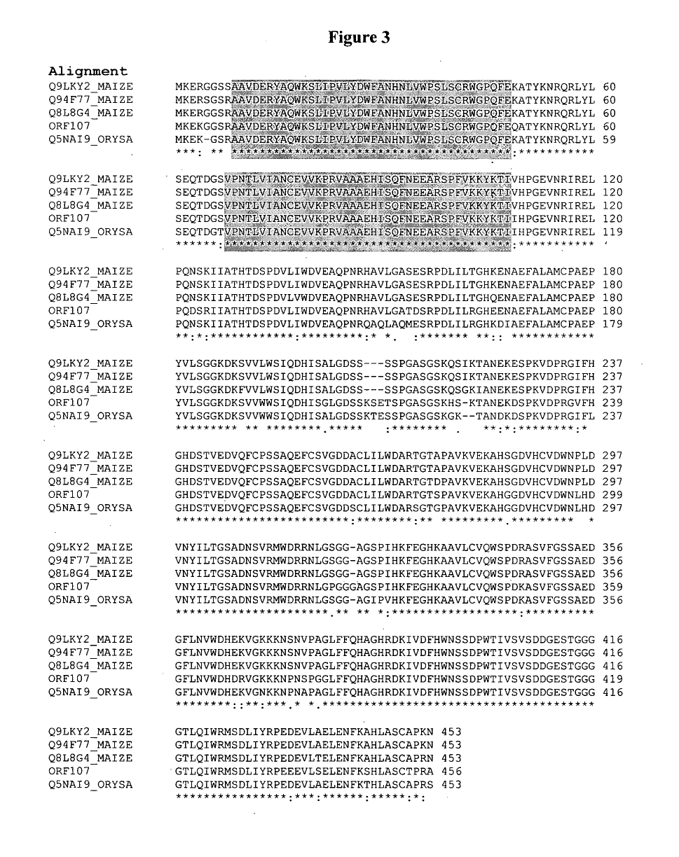

The polypeptide sequence encoded by the ORF107 was used as seed sequence to perform BLASTP search against the UNIPROT database (release 8.2 consists of: Swiss-Prot Protein Knowledgebase Release 50.2 of 27 Jun. 2006 and TrEMBL Protein Database Release 33.2 of 27 Jun. 2006) to identify variants of ORF107. The BLASTP output summary is shown in FIG. 1. A cut-off e value of less than or equal to zero, was identified as distinguishing variants of ORF107 from less related proteins. The selected variant sequences were aligned using the EMBOSS tool EMMA (Thompson et al 1994), which is an interface to the popular multiple alignment program ClustalW. Aligned sequences were visualised using another EMBOSS tool called Prettyplot as shown in FIG. 2. Selected monocot variants were also aligned as shown in FIG. 3.

The variant polypeptide sequences of ORF 107 are listed as SEQ ID NO:2-9 in the sequence listing. The corresponding polynucleotide sequences are listed as SEQ ID NO: 13-21 respectively.

A first consensus polypeptide sequence motif present in ORF107 and all of the selected variants of ORF 107 from monocotyledonous species was identified by the applicants and is highlighted in FIG. 2. The sequence of this conserved motif is shown in SEQ ID NO:10.

A second consensus polypeptide sequence motif present in ORF107 and all of the selected variants of ORF 107 from monocotyledonous species was identified by the applicants and is highlighted in FIG. 2. The sequence of this conserved motif is shown in SEQ ID NO:11.

A third consensus polypeptide sequence motif present in all of the selected variants of ORF 107 from dicotyledonous species was identified by the applicants and is highlighted in FIG. 2. The sequence of this conserved motif is shown in SEQ ID NO:23.

A fourth consensus polypeptide sequence motif present in all of the selected variants of ORF 107 from dicotyledonous species was identified by the applicants and is highlighted in FIG. 2. The sequence of this conserved motif is shown in SEQ ID NO:24.

A fifth consensus polypeptide sequence motif present in ORF107 and all of the selected variants of ORF 107 from plant species was identified by the applicants and is highlighted in FIG. 2. The sequence of this conserved motif is shown in SEQ ID NO:25.

### Construction of a Vector for Over-Expression of ORF107 via Plant Transformation

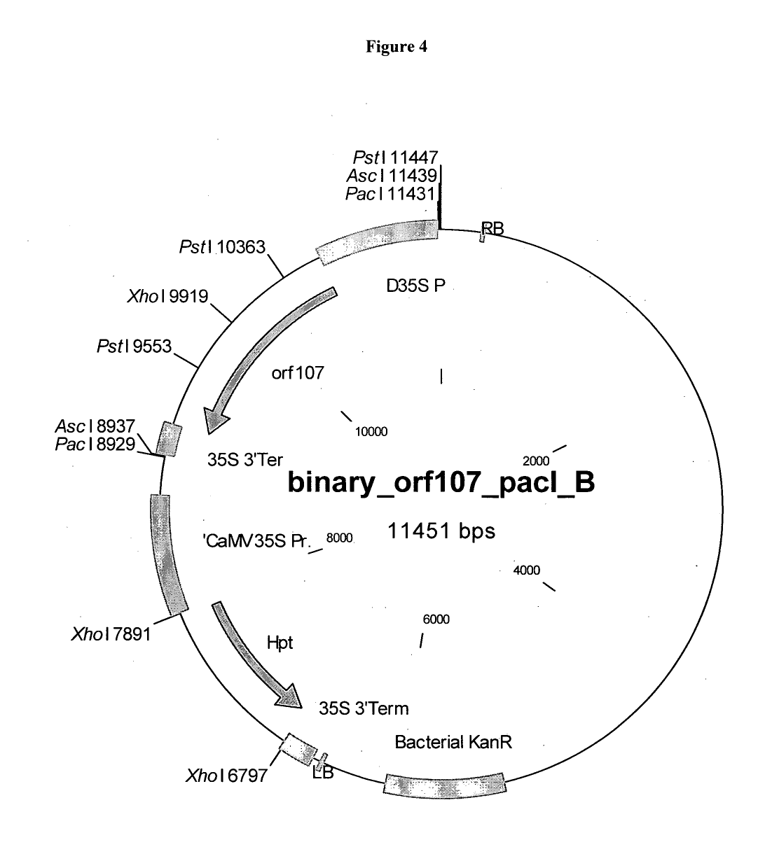

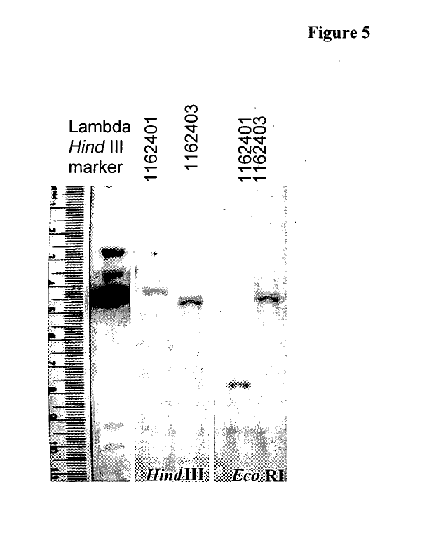

A vector for over-expression ORF107 was produced by standard molecular biology techniques. A map of the binary vector is shown in FIG. 4. The sequence of the vector is shown in SEQ ID NO: 22. Nucleotide start and end positions for various features of the vector are shown below:

| Start | End | Name | Description |
|---|---|---|---|
| 261 | 286 | RB | Right border |
| 5313 | 6095 | Bacterial KanR | Kanamycin resistance gene |
| 6520 | 6545 | LB | Left border |
| 6810 | 6595 | CaMV | CaMV 35S 3′terminator |
| 7861 | 6839 | Hpt | Hygromycin phosphotransferase gene |
| 8678 | 7897 | CaMV35S Pr. | CaMV 35S 3 promoter |
| 9150 | 8945 | 35S 3′Ter | CaMV 35S 3 Termintor |
| 10624 | 9158 | orf107 | Sequence encoding ORF107 |
| 11426 | 10631 | CaMV D35S P | CaMV Double35S Promoter |

### Plant Transformation—Rice

Agrobacterium tumefaciens strains can be transformed with binary plasmid DNA using either a freeze/thaw (Chen et. al 1994) or electroporation method (den Dulk-Ras A and Hooykaas P J.). Purified plasmid DNA of ORF107 was introduced into Agrobacterium strain EHA105 by electroporation and the suspension was incubated at 26° C. for 30 minutes. A small aliquot was plated on AB minimal medium (Schmidt-Eisenlohr et. al 1999) containing Kanamycin at 100 mg/L. Plates were incubated at 26° C. for 3 days and single colonies were tested for presence of the plasmid using construct specific primers and transformation confirmed.

Agrobacterium cultures were grown in AG minimal medium containing 100 mg/L kanamycin at 26° C. with shaking (200 rpm). The Agrobacterium suspensions were pelleted at 5,000 rpm for 5 minutes, washed once in basal MS medium containing 1% glucose and 3% sucrose, pH 5.2, and re-suspended in same medium containing 200 μM acetosyringone to OD₆₀₀ 0.6-0.8.

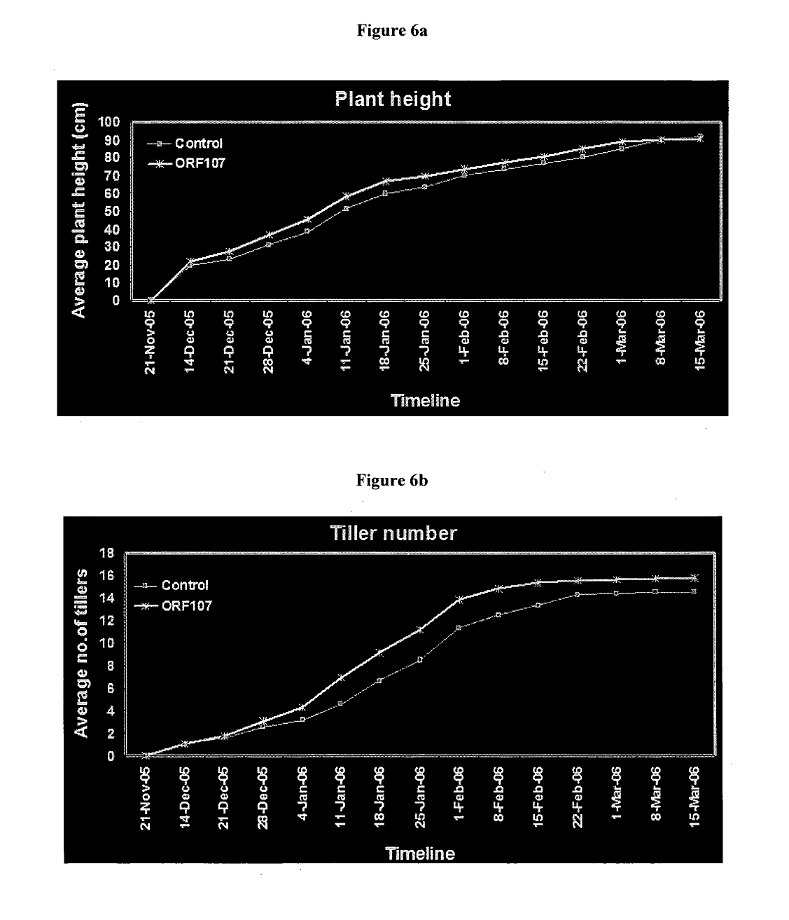

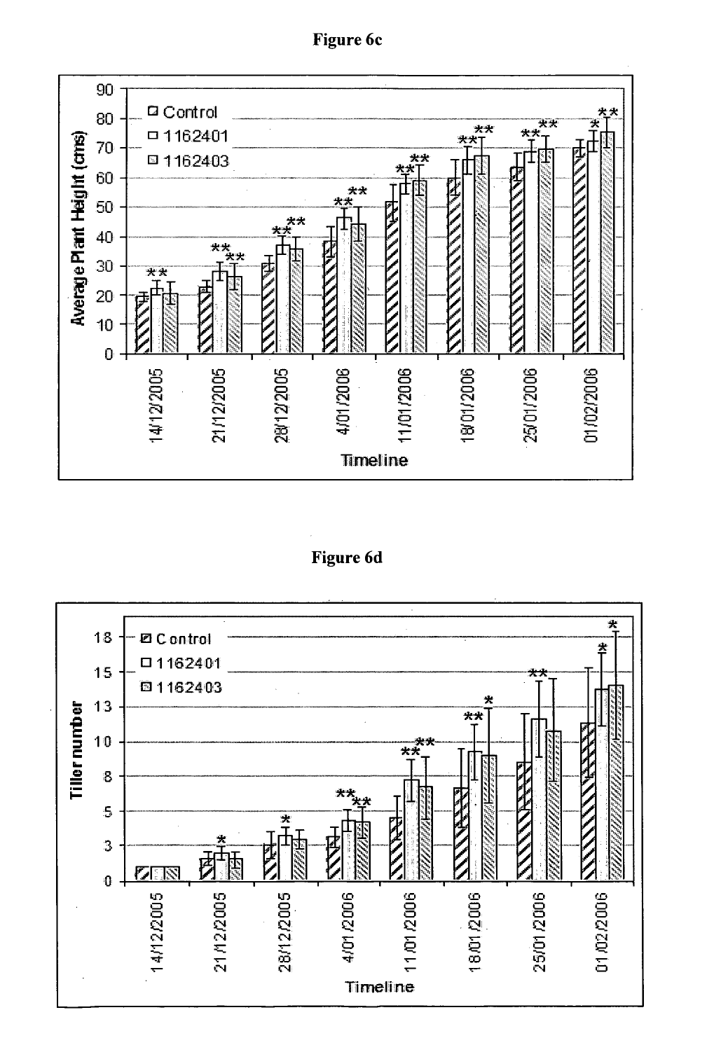

A. tumefaciens containing the binary vector ORF107 were used to co-cultivate at least 1,000 immature rice (Oryza sativa) cv. Nipponbare embryos. Immature seeds from rice were washed in sterile water and then surface sterilized with sodium hypochlorite containing 1.25% active chlorine with 10 μL Tween® 20 for 20 minutes. After sterilization, the seeds were washed several times with sterile water and blotted dry on sterile filter paper (3M). The seeds were de-husked manually using sterile pair of forceps and the embryo dissected out with sterile knife. The isolated embryos were immersed in Agrobacterium suspension for 30 minutes with continuous shaking at 100 rpm in a 10 mL culture tube. The excess liquid was drained off and the embryos blotted on to sterile filter paper before placing them on to co-cultivation medium containing MS medium (Murashige and Skoog, 1964) supplemented with 3% sucrose, 1% glucose, 2 mg/L 2,4-D, 0.1 mg/L BA, 400 μM acetosyringone, pH 5.2 for 4 days in dark. After co-cultivation, the calli forming embryos were sub-cultured once every two weeks on selection medium consisting of MS medium supplemented with 3% sucrose, 1% glucose, 2 mg/L 2,4-D (2,4-dichlorophenoxy acetic acid), 0.1 mg/L BA (benzyl adenine) and containing 50 mg/L hygromycin and 300 mg/L timentin™ (ticarcillin+clavulanic acid) till at-least 30 healthy calli showing green spots indicative of healthy shoot emergence was achieved. Calli containing the green spots were transferred to selection medium lacking 2,4-D to regenerate a minimum of 10 transformed plants. Regenerated plants were rooted and then transplanted to six inch pots containing soil and plants grown in greenhouse. DNA gel-blot analysis was carried out (FIG. 6) to determine gene copy number and identify five independent transformation events. T1 seeds were harvested from the transformed plants (T0).

### T1 Plant Phenotyping

Thirty seeds from Southern positive T0 plants were sown in individual cups containing cocopeat and twenty healthy plants out of them were transplanted in the green house.

These plants were arranged using a CRD using the random numbers from a random table.

T1 plant phenotyping was carried out on progeny lines from T0 events 1162401 and 1162403 and Nipponbare (a wild-type control).

### Phenotypic Analysis of T0 Lines

The physiological state of T0 plants is presented in Table 1, below.

TABLE 1
Physiological measurements of T0 lines

| T0 | | Productive/ | Plant | |
|---|---|---|---|---|
| Plant ID | Pollen fertility | Total No. of tillers | height | Seed Yield |
| 1162401 | 86.9% | 7/11 | 61.9 | 106 |
| 1162403 | 87.9% | 6/10 | 56.4 | 107 |

### Phenotypic Analysis of T1 Lines

Plants height and tiller numbers were measured once every week post-transplanting until seed set was achieved. FIGS. 6a, b, c and d depict the growth parameters observed for these plants. Transgenic ORF107 rice plants (T1) were significantly different in terms of plant height and tillering capacity based upon standard statistical analysis performed on biometric parameters obtained till mid-tillering phase. Transgenic ORF107 rice plants can be said to be normal in all aspects assessed at maturity (data not shown) except for tillering time.

### Plant Transformation—Perennial Ryegrass

Perennial ryegrass (Lolium perenne L. cv. Tolosa) was transformed essentially as described in Bajaj et. al. (Plant Cell Reports, 2006, 25: 651-659). Embryogenic callus derived from mersitematic regions of the tillers of selected ryegrass lines and Agrobacterium tumefaciens strain EHA 101 carrying a modified binary vector (ORF 107) was used for transformation experiments. Embryogenic calli were immersed with overnight-grown Agrobacterium cultures for 30 minutes with continuous shaking. Calli resistant to hygromycin were selected after subculturing them on co-cultivation medium for 4 weeks. After selection, the resistant calli were subcultured on regeneration medium every 2 weeks until the plants regenerated. The regenerants that continued to grow after two or three rounds of selection proved to be stable transformants. Each regenerated plant was then multiplied on maintenance medium to produce clonal plantlets and subsequently rooted on MS medium without hormones. A total of 23 rooted plants were regenerated and a rooted plant from each clone was transferred into contained glasshouse conditions while retaining a clonal counterpart in tissue culture as backup.

### Phenotyping of Plants for Evaluating Tillering Abilities in Growth

A plant growth system is built using 500 mm long; 90 mm diameter plastic storm-water pipes. The pipes are placed on a mobile tray and supported at the sides by ropes and metal frame. The tubes are plugged at the bottom with rockwool and progressively filled with washed mortar sand using water to achieve uniform packing. At the center of the open end of each tube a clump of perennial ryegrass (5 tillers) is planted. Twenty three independent transgenic events are evaluated against two wild-type non-transgenic control lines in triplicate. The plants are arranged at random, and grown at 70% relative humidity; 16/8 hours day/night cycle and under 650 μmol.m-2.s-1 light intensity. The plants are irrigated daily once in the morning with 50 mL Hoagland's solution (Hoagland and Arnon, 1938) and again in the afternoon with 50 mL plain water. Tillers are counted once every two weeks for duration of two months. Expression levels of ORF107, are also evaluated using RT-PCR, and compared with the ability to tiller.

### REFERENCES

Adams et al. 1991, Science 252:1651-1656.
Chen H, Nelson R S, Sherwood J L. (1994) Biotechniques; 16 (4): 664-8, 670.
Chen et al. 2002, Nucleic Acids Res. 31:101-105
den Dulk-Ras A, Hooykaas P J. (1995) Methods Mol Biol.; 55: 63-72.
Hoagland, D. R. and D. I. Arnon. 1938. The water culture method for growing plants without soil. California Agr. Expt. Sta. Circ. 347.
Lee et al. 2003, PNAS 99:12257-12262
Lee and Lee, 2003 Plant Physiol. 132: 517-529
Li, D., Roberts, R., WD-repeat proteins: structure characteristics, biological function, and their involvement in human diseases. (2001) Cell. Mol. Life Sci. 58: 2085-2097
Mayer R R, Cherry J H, Rhodes D (1990) Plant Physiol. 94: 796-810.
Murashige T, Skoog F (1962) Physiol Plant 15: 473-497
Patterson B D, Graham D (1987) In (D D Davies ed) “The Biochemistry of Plants”, Vol 12, Academic Press, New York, pp. 153-199.
Richmond and Somerville 2000, Current Opinion in Plant Biology. 3:108-116
Ruan et al. 2004, Trends in Biotechnology 22: 23-30.
Schmidt-Eisenlohr H, Domke N, Angerer C, Wanner G, Zambryski P C, Baron C. (1999) J. Bacteriol.; 181 (24): 7485-92.
Smith, T. F., Gaitatzes, C., Saxena, K., Neer, E. J., The WD repeat: a common architecture for diverse functions. (1999) Trends Biochem. Sci. 24: 181-185
Sun et al. 2004, BMC Genomics 5: 1.1-1.4
Thompson, J. D., Higgins, D. G. and Gibson, T. J. (1994) CABIOS, 10, 19-29.
Velculescu et al. 1995, Science 270: 484-487

The above examples illustrate practice of the invention. It will be appreciated by those skilled in the art that numerous variations and modifications may be made without departing from the spirit and scope of the invention.

Summary of Sequences

| SEQ ID NO. | Sequence type | Species | Reference |
|---|---|---|---|
| 1 | polypeptide | Lolium perenne | ORF 107 |
| 2 | polypeptide | Oryza sativa | Q5NAI9 |
| 3 | polypeptide | Zea mays | Q9LKY2 |
| 4 | polypeptide | Zea mays | Q94F77 |
| 5 | polypeptide | Zea mays | Q8L8G4 |
| 6 | polypeptide | Pisum sativum | Q52ZH8 |
| 7 | polypeptide | Arabidopsis thaliana MSI4 | O22607 |
| 8 | polypeptide | Silene latifolia | Q9ST70 |
| 9 | polypeptide | Silene latifolia | Q8VX59 |
| 10 | polypeptide | Monocot consensus motif #1 | — |
| 11 | polypeptide | Monocot consensus motif #2 | — |
| 12 | polynucleotide | Lolium perenne | ORF107 |
| 13 | polynucleotide | Lolium perenne | ORF107 genomic |
| 14 | polynucleotide | Oryza sativa | AP002901/Q5NAI9 |
| 15 | polynucleotide | Zea mays | AF250047/Q9LKY2 |
| 16 | polynucleotide | Zea mays | AF384037/Q94F77 |
| 17 | polynucleotide | Zea mays | AY100481/Q8L8G4 |
| 18 | polynucleotide | Pisum sativum | AY830931/Q52ZH8 |
| 19 | polynucleotide | Arabidopsis thaliana MSI4 | AF498102/O22607 |
| 20 | polynucleotide | Silene latifolia | Y18519/Q9ST70 |
| 21 | polynucleotide | Silene latifolia | AJ310656/Q8VX59 |
| 22 | polynucleotide | ORF107 binary vector | — |
| 23 | polypeptide | Dicot consensus motif #1 | — |
| 24 | polypeptide | Dicot consensus motif #2 | — |
| 25 | polypeptide | All plant consensus motif | — |
| 26 | polypeptide | Lolium perenne | WD40 motif |
| 27 | polypeptide | Lolium perenne | WD40 motif |
| 28 | polypeptide | Lolium perenne | WD40 motif |
| 29 | polypeptide | Lolium perenne | WD40 motif |
| 30 | polypeptide | Lolium perenne | WD_REPEATS_2 motif |
| 31 | polypeptide | Lolium perenne | WD_REPEATS_2 motif |
| 32 | polypeptide | Lolium perenne | WD_REPEATS_2 motif |
| 33 | polypeptide | Lolium perenne | WD_REPEATS_REGION |
| | | | motif |
| 34 | polypeptide | Lolium perenne | WD40-repeat like motif |
| 35 | polypeptide | Lolium perenne | motif |
| 36 | polypeptide | Lolium perenne | motif |
| 37 | polypeptide | Lolium perenne | motif |

1-37. (canceled)

38. A method for producing a plant with altered tillering time, the method comprising transformation of a plant cell or plant with a:
a) a polynucleotide encoding of a polypeptide with the amino acid sequence of SEQ ID NO: 1 or a variant of the polypeptide;
b) a polynucleotide comprising a fragment, of at least 15 nucleotides in length, of the polynucleotide of a); or
c) a polynucleotide comprising a compliment, of at least 15 nucleotides in length, of the polynucleotide of a).

39. The method of claim 38 wherein the variant in a) is capable of modulating tillering time in a plant.

40. The method of claim 38 in which the variant has at least 70% sequence identity to a polypeptide with the amino acid sequence of SEQ ID NO: 1.

41. The method of claim 38 in which the variant is derived from a plant species and comprises the amino acid sequence of SEQ ID NO: 25.

42. The method of claim 38 in which the variant is derived from a monocotyledonous species and comprises the amino acid sequence of at least one of SEQ ID NO: 10 and SEQ ID NO: 11.

43. The method of claim 38 in which the variant is derived from a dicotyledonous species and comprises the amino acid sequence of at least one of SEQ ID NO: 23 and SEQ ID NO: 24.

44. The method of claim 38 in which the variant comprises an amino acid sequence selected from any one of SEQ ID NO: 2-9.

45. The method of claim 38 in which the polynucleotide of a) encodes a polypeptide with the amino acid sequence of SEQ ID NO: 1.

46. The method of claim 38 in which the polynucleotide of a) comprises a sequence with at least 70% sequence identity to SEQ ID NO: 12.

47. The method of claim 38 in which the polynucleotide of a) comprises the sequence of any one of SEQ ID NO: 14 to 21.

48. The method of claim 38 in which the polynucleotide of a) comprises the coding sequence of any one of SEQ ID NO: 14 to 21.

49. The method of claim 38 in which the polynucleotide of a) comprises the sequence of SEQ ID NO: 12.

50. The method of claim 38 in which the polynucleotide of a) comprises the coding sequence of SEQ ID NO: 12.

51. The method of claim 38 in which the plant with altered tillering time is early tillering relative to a suitable control plant.

52. The method of claim 38 in which the plant with altered tillering time is late tillering relative to a suitable control plant.

53. A plant cell or plant produced by a method of claim 38.

54. An isolated polynucleotide encoding a polypeptide with at least 70% identity to the amino acid sequence selected of SEQ ID NO: 1.

55. The isolated polynucleotide of claim 54 wherein the polypeptide is capable of modulating tillering time in a plant.

56. The isolated polynucleotide of claim 54 comprising the sequence of SEQ ID NO: 12.

57. The isolated polynucleotide sequence of claim 54 comprising the full-length coding sequence of SEQ ID NO: 12.

58. The isolated polynucleotide sequence of claim 54 comprising the sequence of SEQ ID NO: 13.

59. The isolated polynucleotide sequence of claim 54 comprising a sequence with at least 70% identity to SEQ ID NO: 12.

60. The isolated polynucleotide sequence of claim 54 comprising the sequence of SEQ ID NO: 12.

61. An isolated polypeptide having at least 70% sequence identity to the amino acid sequence of SEQ ID NO: 1.

62. The isolated polypeptide of claim 61, wherein the polypeptide is capable of modulating tillering time in a plant.

63. The isolated polypeptide of claim 61 comprising the amino acid sequence of SEQ ID NO: 1.

64. A genetic construct which comprises the polynucleotide of claim 54.

65. A genetic construct including a polynucleotide consisting of at least one of:
a) a fragment, of at least 15 nucleotides in length, of a polynucleotide of claim 54;
b) a complement, of at least 15 nucleotides in length, of the polynucleotide of claim 54; or
c) a sequence, of at least 15 nucleotides in length, capable of hybridising to the polynucleotide of claim 54.

66. A host cell, plant cell, or plant genetically modified to express a polynucleotide of claim 54.

67. A host cell, plant cell, or plant genetically modified to express a polypeptide of claim 61.

68. A host cell, plant cell, or plant comprising a genetic construct including a polynucleotide consisting of at least one of:
a) a fragment, of at least 15 nucleotides in length, of a polynucleotide of claim 54;
b) a complement, of at least 15 nucleotides in length, of the polynucleotide of claim 54; or
c) a sequence, of at least 15 nucleotides in length, capable of hybridising to the polynucleotide of claim 54.

69. A method for selecting a plant with altered tillering time relative to suitable control plant, the method comprising testing of a plant for altered expression of a polynucleotide of claim 54.

70. A method for selecting a plant with altered tillering time relative to suitable control plant, the method comprising testing of a plant for altered expression of a polypeptide of claim 61.

71. An antibody raised against a polypeptide of claim 61.
